(19) United States
(12) Patent Application Publication
(10) Pub. No.: US 2019/0160021 A1
(43) Pub. Date: May 30, 2019

(54) Substituted Stilbenes as Inhibitors of NF-kappaB and Activators of Nrf2

(71) Applicant: STC.UNM, ALBUQUERQUE, NM (US)

(72) Inventors: David L. Vander Jagt, Albuquerque, NM (US); Lorraine M. Deck, Albuquerque, NM (US); Robert E. Royer, Albuquerque, NM (US)

(21) Appl. No.: 16/204,085

(22) Filed: Nov. 29, 2018

Related U.S. Application Data
(60) Provisional application No. 62/591,912, filed on Nov. 29, 2017.

(51) Int. Cl. A61K 31/035; A61K 31/09; A61K 31/05; A61K 31/122; A61K 45/06; A61P 35/00; A61P 17/00

(52) U.S. Cl. CPC A61K 31/035; A61K 31/09; A61K 31/05; A61P 17/00; A61K 45/06; A61P 35/00; A61K 31/122

(57) ABSTRACT

The present invention relates to substituted stilbenes and dienones which exhibit unexpected dual activity, as inhibitors of NFκB and as agonists (activators) of Nrf2. In particular, these compounds show dual activity and makes them particularly useful in the treatment of inflammation, including chronic inflammation and a number of related chronic disease states and conditions, including neurological diseases, including Alzheimer's, Alzheimer's prodrome and mild cognitive impairment, and other diseases and conditions, such as Parkinson's disease, depression, bipolar disorders and autism spectrum disorders. Compounds, pharmaceutical compositions and methods of treatment are described.

### RELATED APPLICATIONS

This application claims the benefit of priority of provisional application serial number U.S. 62/591,912, of identical title, filed 29 Nov. 2017, the entire contents of which is incorporated by reference herein.

### FIELD OF THE INVENTION

The present invention relates to substituted stilbenes and dienones which exhibit unexpected activity, as inhibitors of proinflammatory NFκB signaling and as agonists (activators) of anti-oxidant Nrf2 signaling. In particular, these compounds show dual anti-inflammatory activity and anti-oxidant activity, which makes them particularly useful in the treatment of inflammation and its associated oxidative stress, including chronic inflammation and a number of related chronic disease states and conditions, including neurological diseases, including Alzheimer's, Alzheimer's prodrome and mild cognitive impairment, and other diseases and conditions, such as Parkinson's disease, depression, bipolar disorders and autism spectrum disorders. Compounds, pharmaceutical compositions and methods of treatment are described.

### BACKGROUND AND OVERVIEW OF THE INVENTION

The nuclear factor κB (NF-κBa) family of transcription factors in mammals consists of homo- and heterodimeric combinations of five related proteins (p50, p52, p65/RelA, c-Rel, and RelB) that have a marked influence on the expression of numerous genes involved in immunity and inflammation, as well as cellular stress responses, growth, and apoptosis. Diverse pathways activate NF-κB, and control of these pathways is increasingly viewed as an approach to chemotherapy in the many diseases that have an associated inflammatory component, including cancer, stroke, Alzheimer s disease, and diabetes. Activation of NF-κB occurs through multiple pathways. The classical pathway is triggered by binding of proinflammatory cytokines (TNFα and IL-1) and of a number of pathogens to several different receptors in the TNF-receptor and Toll-like/IL-1 receptor superfamilies. This leads to recruitment to the plasma membrane and activation of the IκB-kinase complex (IKK) consisting of IKKα and IKKβ kinases, and the scaffold protein NEMO/IKKγ, as well as a number of IKK-associated proteins. The main NF-κB that is activated in the classical pathway is the p50/p65 heterodimer that exists in the cytoplasm as a complex with inhibitory protein IκBα. Activation of IKK primarily through IKKβ results in phosphorylation of IκBα on Ser32 and Ser36, followed by polyubiquitination and degradation of IκBα by the 26S proteasome, allowing p50/p65 to translocate to the nucleus.

Release of p50/p65 from IκBα also can be achieved by IKK-independent pathways triggered by DNA damage or oxidative stress that result in phosphorylation of IκBα on Ser residues other than Ser32 or Ser36, again leading to proteosomal degradation of IκBα. This signaling pathway involves a number of kinases including the MAP kinase p38 and casein kinase 2. There is also an oxidative stress pathway that phosphorylates IκBα on Tyr residues, leading to release of p50/p65 without proteosomal degradation of IκBα. Superimposed on the complex activation of p50/p65 is additional downstream regulation of the DNA-binding properties of p50/p65 through phosphorylation, acetylation, and peptidyl-prolyl isomerization. Mostly this occurs in p65 and provides multiple points for control of NF-κB activation in a cell-specific and environment-specific manner. A wide range of kinases can phosphorylate p50/p65, which appears essential for the transactivation potential of p50/p65. This includes phosphorylation at many different sites, especially in p65, which adds to the complex regulation of NF-κB.

Transcription factor nuclear factor erythroid 2 related factor 2 (Nrf2), which is a member of the cap‘n’collar family of transcription factors, is the master regulator of an inducible cellular system of cytoprotective genes. These genes code for a broad range of proteins, including phase I and 11 detoxification enzymes, anti-oxidant proteins, as well as anti-inflammatory and neuroprotective factors, growth factors and receptors, and other transcription factors. Interest has emerged in Nrf2 as a therapeutic target, especially for treatment of chronic inflammatory diseases and the associated oxidative stress.¹⁻⁶ In the absence of stress, Nrf2 forms a cytosolic complex with Kelch-like ECH associated protein 1 (Keap1) and Cul3; Cul3 is an adaptor to link Nrf2 to an E3 ubiquitin ligase complex. Newly synthesized Nrf2 in unstressed cells is degraded by ubiquitination and proteosomal degradation, which limits the cytosolic concentration of Nrf2. In response to oxidative and electrophilic stresses, cysteine residues of Keap1 are modified, which alters the interaction between Nrf2 and Keap1 and locks Keap1 in the Nrf2 complex. This allows newly synthesized Nrf2 to accumulate and translocate to the nucleus where Nrf2 interacts with small Maf proteins and then binds to promoters with anti-oxidant response element (ARE) sequences.⁷⁻¹¹

Numerous Nrf2-activating chemicals have been identified, including some natural product phenols, such as the enone curcumin and the trans stilbene resveratrol, that can activate Nrf2 after oxidation to electrophilic quinones, which can modify select cysteine residues in Keap1 by Michael addition. Keap1 cysteine residues 273, 288 and 151 appear to be especially important.^{12,13} Peptide inhibitors of the Keap1-Nrf2 protein-protein interaction have been developed as well as a variety of small molecules that inhibit the Keap1-Nrf2 interaction.¹⁴⁻¹⁹ These studies have been aided by the availability of several crystal structures of Nrf2.²⁰⁻²²

Natural product phenols such as curcumin and resveratrol exhibits numerous biological activities including ability to induce the expression of Nrf2-dependent phase II and anti-oxidant enzymes such as glutathione S-transferase, aldose reductase and heme oxygenase-1.²³⁻²⁵ Curcumin appears to utilize more than a single mechanism for activation of Nrf2, including covalent modification of Keap1^{25,26} and activation of upstream kinases.^{25,27} Curcumin has been examined in a number of clinical studies with limited success,²⁸ mainly owing to limited bioavailability and rapid metabolism. Attempts to improve curcumin as a therapeutic agent include development of new formulations that may enhance bioavailability.

There is considerable interest in the development of analogues and derivatives of natural product phenols with improved therapeutic potential.²⁹⁻³² There also is interest in the development of analogues that activate anti-oxidant Nrf2 but simultaneously inhibit pro-inflammatory NF-κB signaling,³³⁻³⁵ which is consistent with the ability of curcumin and resveratrol to target both of these pathways.^{36,37} In the present application is a description and evaluation of the Nrf2-activating potential and NF-κB inhibiting potential of trans stilbene analogues of resveratrol as well as dienone analogues of curcumin.

NF-κB is a major pro-inflammatory transcription factor. Nrf2 is a major anti-oxidant transcription factor, which also controls anti-inflammatory and neuroprotective genes. The importance of inflammation and oxidative stress in many chronic diseases supports the concept that simultaneous inhibition of NF-κB signaling and activation of anti-oxidant Nrf2 signaling may have therapeutic potential. A number of Nrf2 activators have entered into clinical trials. One concern with the design of Nrf2 activators that are electrophilic covalent modifiers of Keap1 is the issue of selectivity. In the present report, substituted trans stilbenes were identified as activators of Nrf2. These activators of Nrf2 are not highly electrophilic and therefore are unlikely to activate Nrf2 through covalent modification of Keap1. Dose-response studies demonstrated that a range of substituents on either ring of the trans stilbenes, especially fluorine and methoxy substituents, influenced not only the sensitivity to activation, reflected in EC₅₀ values, but also the extent of activation, which suggests that multiple mechanisms are involved in the activation of Nrf2. The stilbene backbone appears to be a privileged scaffold for development of a new class of Nrf2 activators. In addition, many of these trans stilbenes analogues of resveratrol are also inhibitors of the activation of NF-κB. Moreover, a number of dienone analogues of curcumin are described that also exhibit this dual activity.

### SUMMARY OF THE INVENTION

Inasmuch as inflammation and oxidative stress are often inseparable in a large number of disease states, compounds which affect both of these physiological manifestations simultaneously represent particularly effective compounds for use in treating any disease state or condition which would benefit from the simultaneous inhibition of the NF-κB pathway and activation of the Nrf2 pathway. Accordingly, compounds according to the present invention may function as particularly effective therapy in disease states and/or conditions where the NFκB pathway is upregulated and/or stimulated and the Nrf2 pathway is down regulated or inhibited.

In one embodiment, the present invention is directed to a compound according to the chemical structure which appears below. All of these compounds exhibit dual activity as inhibitors (down regulators) of the NF-κB pathway and agonists (up-regulators) of the Nrf2 pathway.

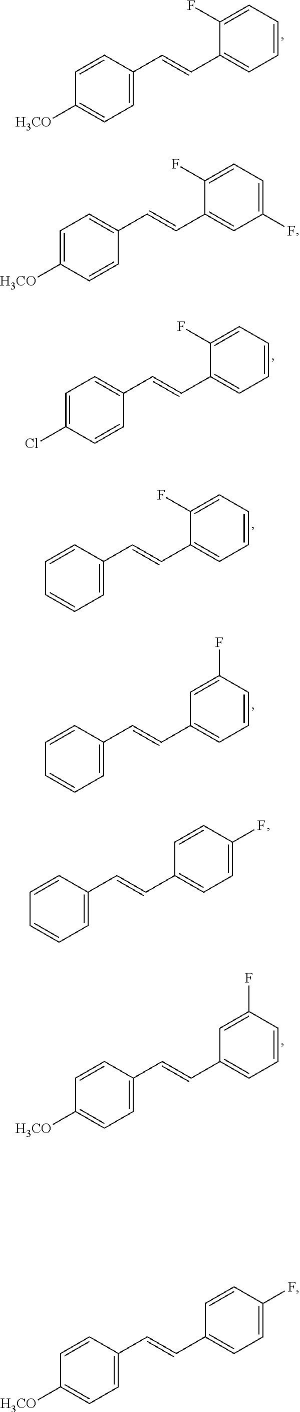

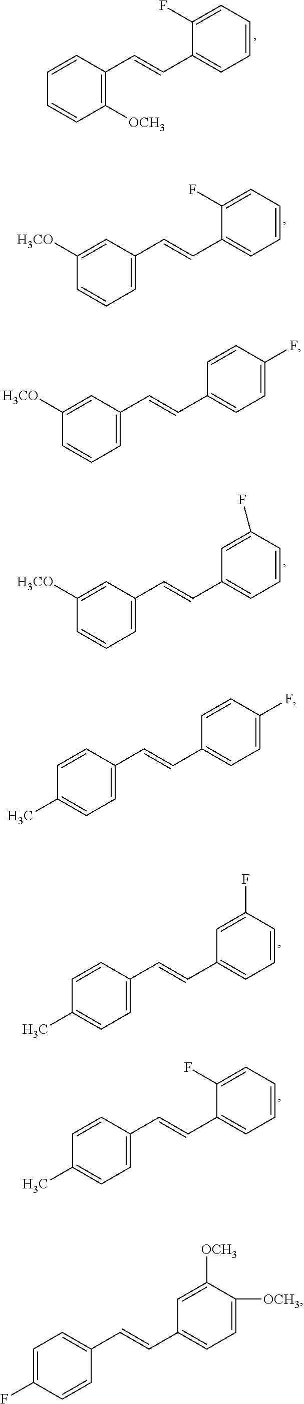

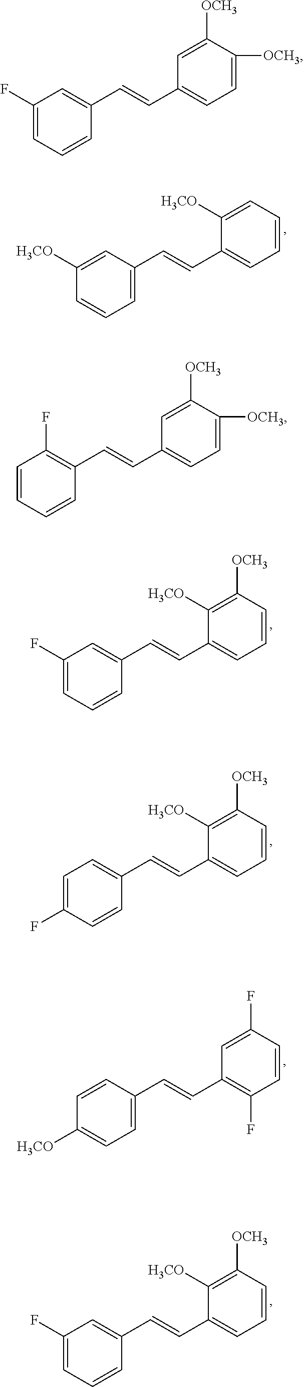

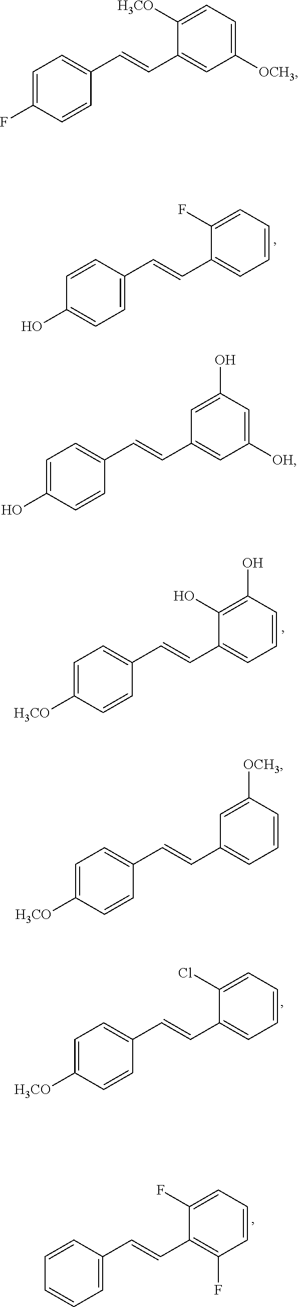

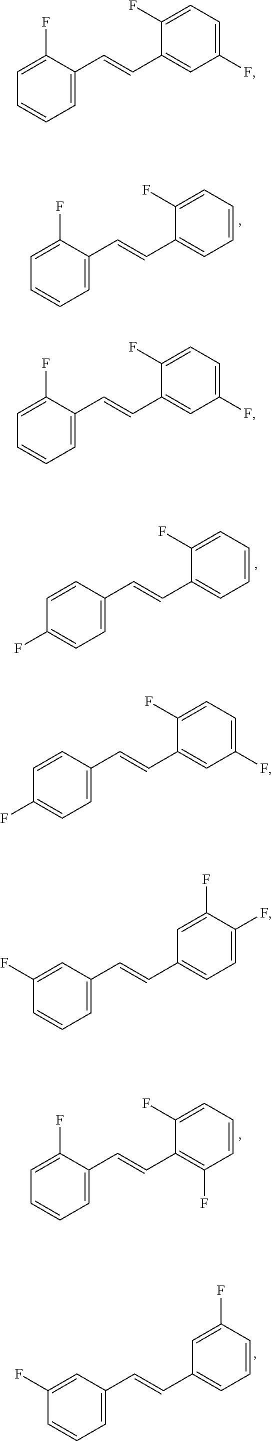

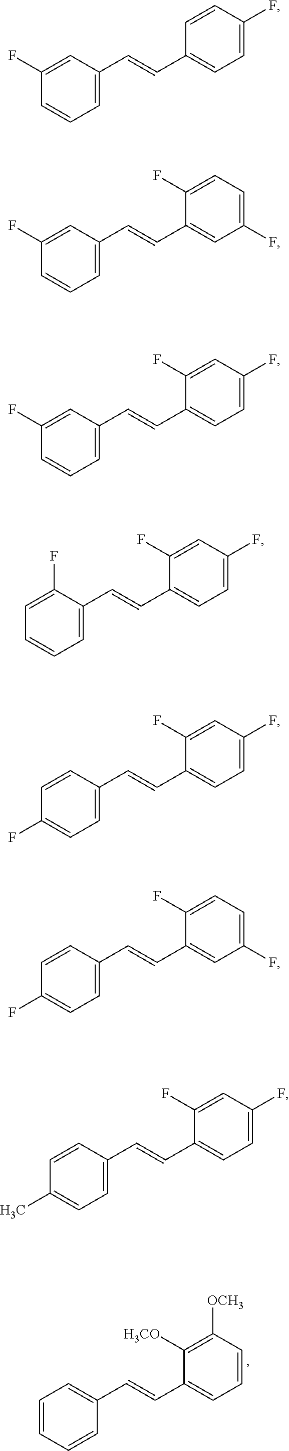

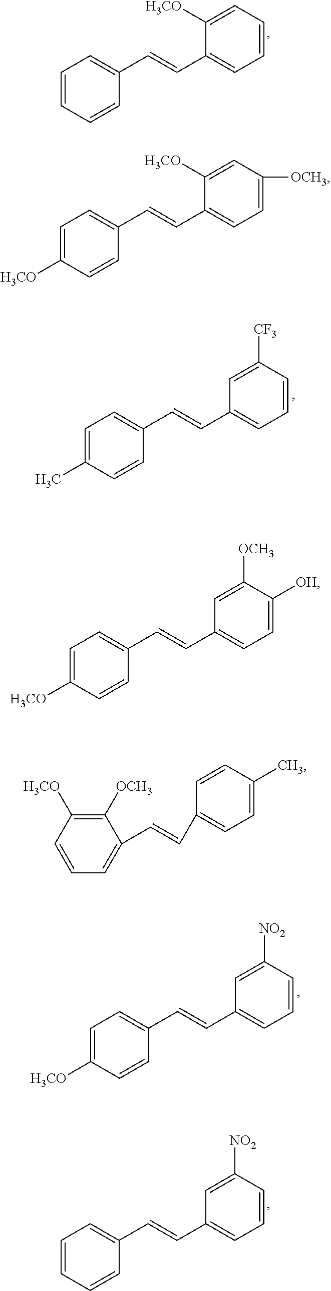

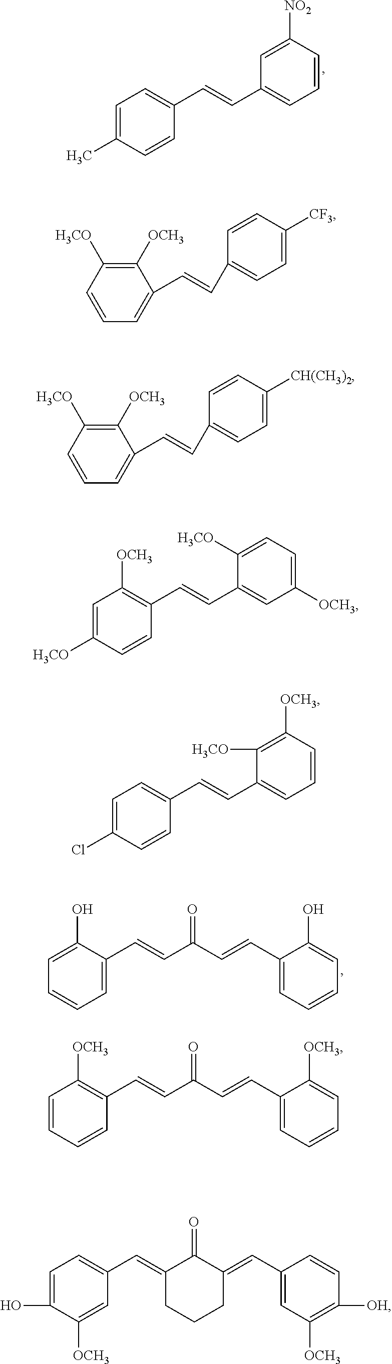

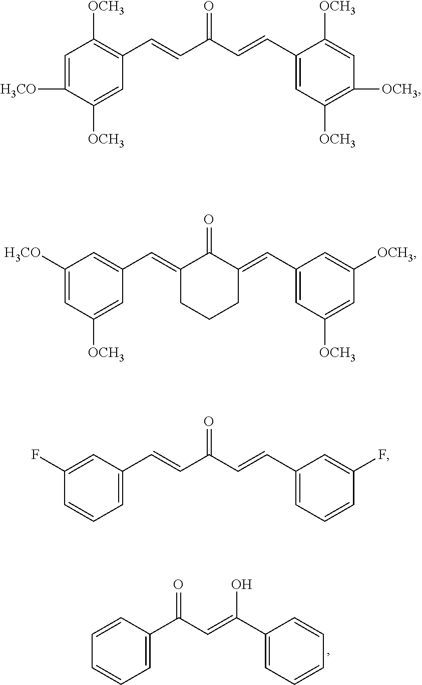 [SMILES: COc1cc(OC)c(OC)cc1/C=C/C(=O)/C=C/c1cc(OC)c(OC)cc1OC ; COc1cc(/C=C2\CCC/C(=C\c3cc(OC)cc(OC)c3)C2=O)cc(OC)c1 ; O=C(/C=C/c1cccc(F)c1)/C=C/c1cccc(F)c1 ; O=C(/C=C(\O)c1ccccc1)c1ccccc1]

or
a pharmaceutically acceptable salt thereof.

Preferred compounds according to the present invention include the following compounds:

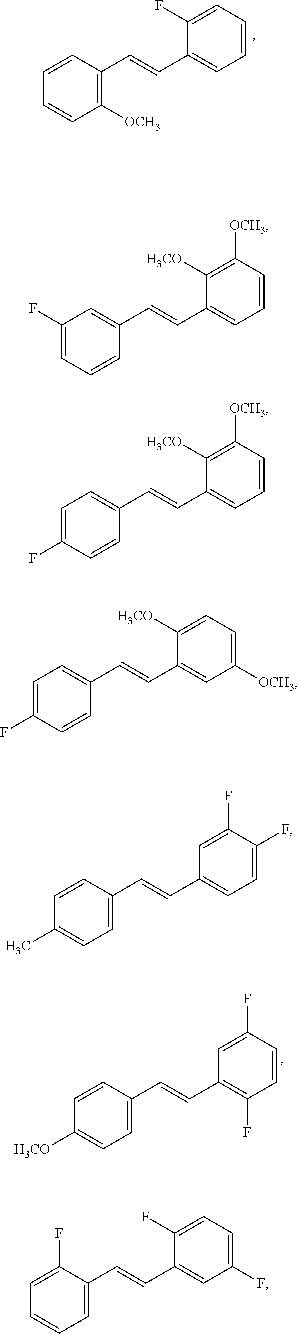

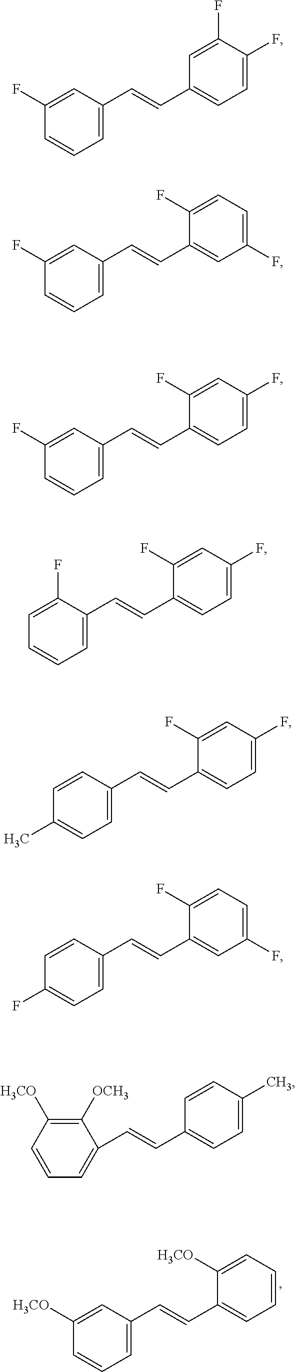

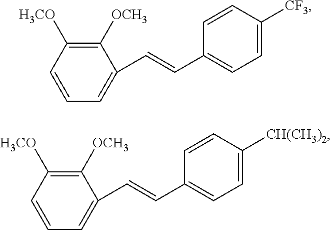

or a pharmaceutically acceptable salt thereof.

In alternative embodiments, preferred compounds include the following compounds:

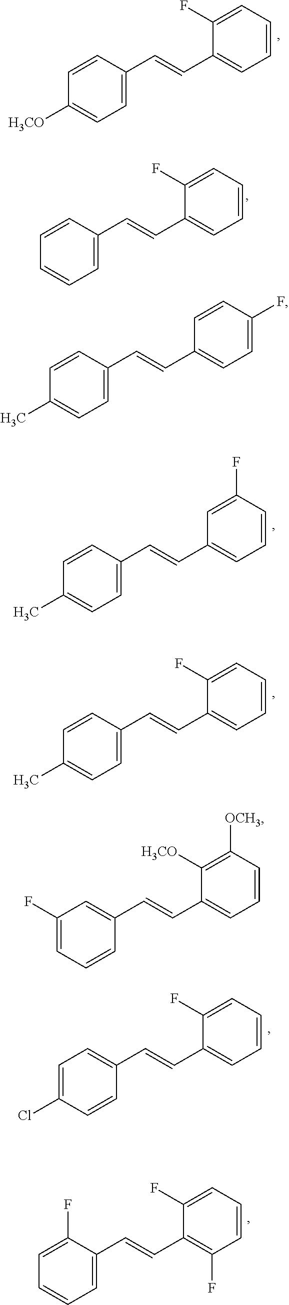

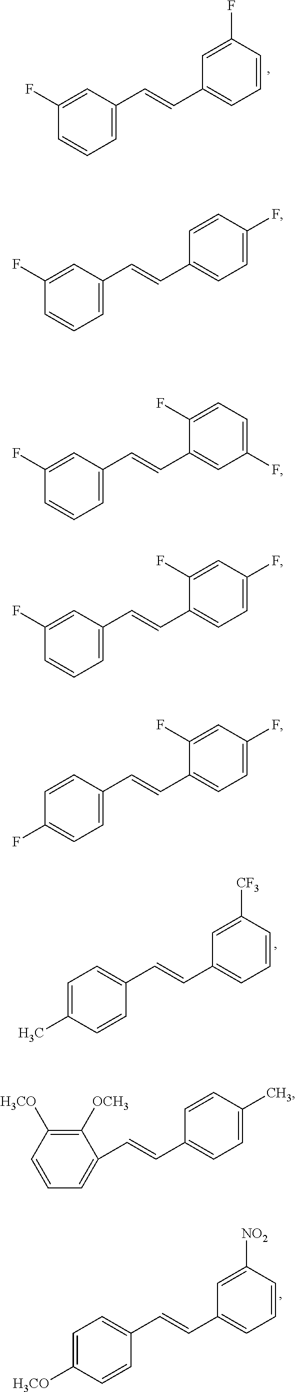

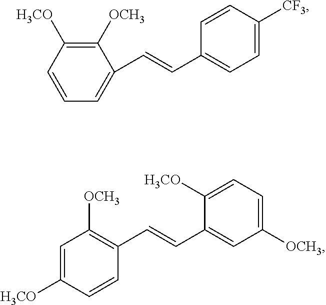 [SMILES: COc1cccc(/C=C/c2ccc(C(F)(F)F)cc2)c1OC ; COc1ccc(OC)c(/C=C/c2ccc(OC)cc2OC)c1]

or
a pharmaceutically acceptable salt thereof.

In another embodiment, the present invention relates to pharmaceutical compositions comprising an effective amount of at least one compound according to the chemical structure set forth above in combination with a pharmaceutically acceptable carrier, additive or excipient, optionally in combination with an additional bioactive agent. In certain embodiments, pharmaceutical compositions comprise at least two of the above-described compounds.

In additional embodiments, the present invention is directed to pharmaceutical compositions comprising an effective amount of at least one compound according to the chemical structure:

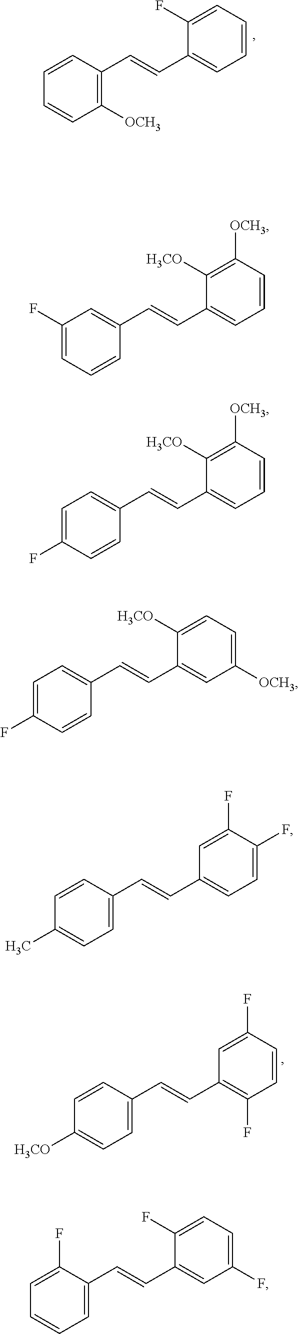

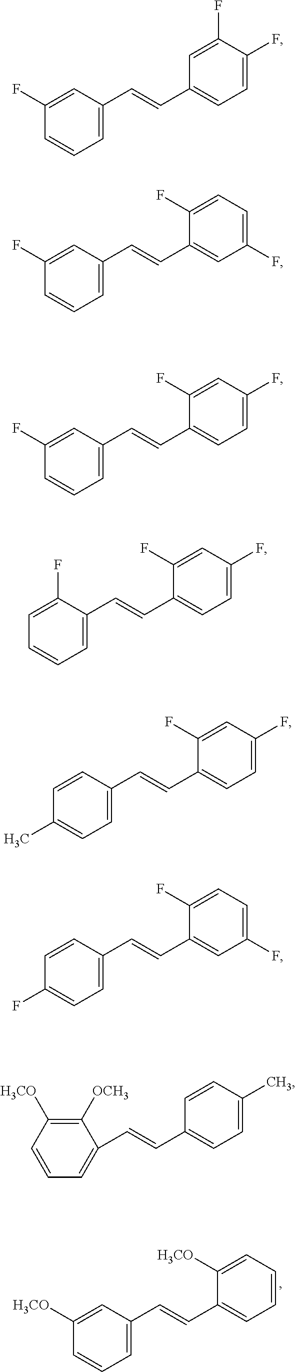

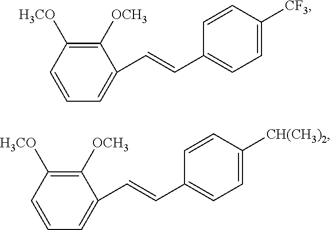

or a pharmaceutically acceptable salt thereof in combination with a pharmaceutically acceptable carrier, additive or excipient, optionally in combination with an additional bioactive agent. In certain embodiments, pharmaceutical compositions comprise at least two of the above-described compounds in a single composition, optionally in combination with an additional bioactive agent.

In additional embodiments, the present invention is directed to pharmaceutical compositions comprising an effective amount of at least one compound according to the chemical structure:

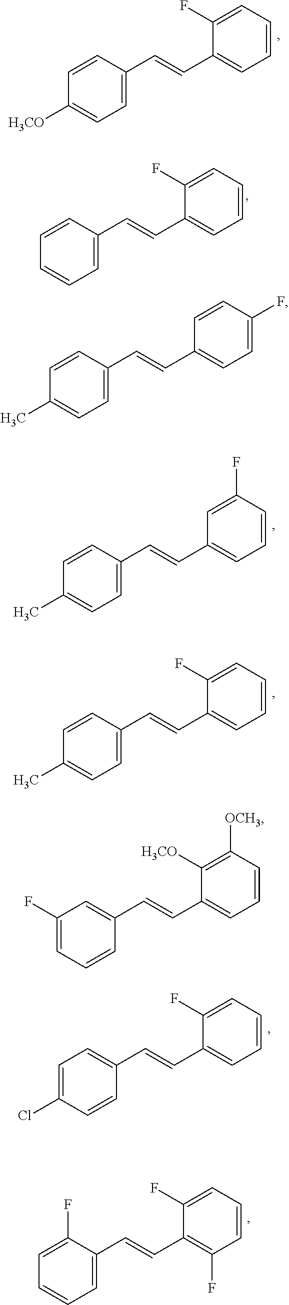

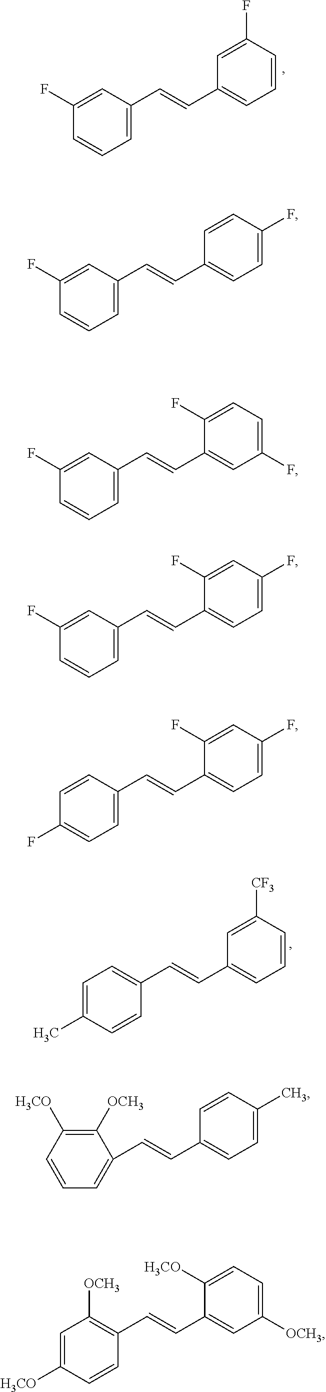

or
a pharmaceutically acceptable salt thereof in combination with a pharmaceutically acceptable carrier, additive or excipient, optionally in combination with an additional bioactive agent. In certain embodiments, pharmaceutical compositions comprise at least two of the above-described compounds in a single composition.

In still another embodiment, the present invention is directed to a method for treating or reducing the likelihood of a disease state or condition which is modulated through NF-κB (by up regulation) and Nrf2 (by down regulation) such that compounds which inhibit NF-κB signaling and increase Nrf2 signaling find particular use in the treatment of these disease states and/or conditions. These disease states and/or conditions include for example, Alzheimer's; ALS; autism; bipolar disorder; brain injury; chronic pain; chronic inflammatory demyelinating polyneuropathy (CIPD); diabetic neuropathy; epilepsy; fibromyalgia; Huntington's; inflammatory myopathy; meningitis; migraine; multiple sclerosis (MS); Parkinson's; stroke; chronic kidney disease; Crohn's disease; diabetes mellitus type 1 and type 2; rheumatoid arthritis, osteoarthritis, psoriatic arthritis, lupus, gout, ulcerative colitis; acne; eczema; psoriasis; tendinitis; atherosclerosis; obesity; cancer, allergies; depression, among others, including skin diseases or conditions as described herein. In a particular embodiment of the present invention, an effective amount of at least one compound set forth above, optionally in combination with an additional bioactive agent is administered to a patient in need to treat one or more of these disease states. In additional embodiments, one or more compounds or compositions disclosed herein may be used in inhibit the NF-κB pathway and enhancing (up-regulate) the Nrf2 signaling pathway to provide particularly effective therapy.

It should be understood that the method of the invention is generally useful for treating any disease state or condition that can be ameliorated by both inhibiting (down-regulating) the NF-κB pathway and enhancing (up-regulating) the Nrf2 signaling pathway to provide particularly effective therapy.

### BRIEF DESCRIPTION OF THE FIGURES

FIG. 1 shows scheme 1, which is directed to the chemical synthesis of compounds according to the present invention. In scheme I, a substituted phosphonate ester is reacted with a substituted aldehyde under reaction conditions a to produce the substituted stilbene compound. The reaction conditions a employed are: (a) R₁ substituted phosphonate ester (1.5 equiv), NaH (2 equiv), dry THF, 0° C., 30 min, R₂ substituted aldehyde (1.0 equiv), 0° C. to rt, 20 h, then H2O/HCl; see FIG. 2, Table 1.

FIG. 2 shows a number of the compounds according to the present invention and references for their synthesis.

FIGS. 3-5 show a number of compounds according to the present invention and their Nfr2 activation activity.

FIG. 6 shows a number of compounds according to the present invention and their NF-κB inhibition activity.

### DETAILED DESCRIPTION OF THE INVENTION

Unless otherwise specified, “a,” “an,” “the,” and “at least one” are used interchangeably and mean one or more than one, depending on the context of use.

The following terms shall be used to describe the present invention. In instances where a term is not defined herein, such term is given its common meaning by those of ordinary skill in the art.

The term “patient” or “subject” refers to a mammal, preferably a human, including a domesticated mammal (including a dog, cat, sheep, horse, cow, pig, goat or other domesticated mammal in need of treatment or therapy to which compounds according to the present invention are administered in order to treat a condition or disease state otherwise described herein.

The term “compound”, as used herein, unless otherwise indicated, refers to any specific chemical compound disclosed herein and includes within context, tautomers, regioisomers, geometric isomers, and where applicable, optical isomers thereof where applicable, as well as pharmaceutically acceptable salts, solvates and polymorphs thereof. Within its use in context, the term compound generally refers to a single compound, but also may include other compounds such as stereoisomers, regioisomers and/or optical isomers (including in some instances, racemic mixtures) as well as specific enantiomers or enantiomerically enriched mixtures of disclosed compounds. The compounds of this invention include all pharmaceutically acceptable salt forms, solvates, polymorphs and prodrug forms of the present compounds, where applicable. The present invention relates to both the cis- and trans-stilbene structures, preferably, trans structures as generally presented herein and their methods of use.

The term “modulate” means, with respect to disease states or conditions, modulated through (e.g. by binding) or having an effect on NF-κB and/or Nrf2 signaling pathways to produce, either directly or indirectly, an improvement or lessening of a condition or disease state which was, prior to administration of a compound according to the present invention, sub-optimal and in many cases, debilitating and even life threatening. Modulation occurs by virtue of antagonist/inhibitor activity for NF-κB and agonist activity for Nrf2 signalling pathway activity. In most/many instances, the term modulate shall mean direct or indirect inhibition or enhancement/up-regulation of NF-κB/Nrf2 signalling pathways alone or within the context of treating a disease or condition associated with same.

The term “independently” is used herein to indicate that the variable, which is independently applied, varies independently from application to application.

The term “disease or condition” is used to describe disease states or conditions which are modulated through NF-κB signaling pathways (via up regulation) and Nrf2 signaling pathways (via down regulation) such that compounds which both inhibit NF-κB signaling and increase Nrf2 signaling may be used to treat these disease states and/or conditions. These disease states and/or conditions include for example, Alzheimer's; ALS; autism; bipolar disorder; brain injury; chronic pain; chronic inflammatory demyelinating polyneuropathy (CIPD); diabetic neuropathy; epilepsy; fibromyalgia; Huntington's; inflammatory myopathy; meningitis; migraine; multiple sclerosis (MS); Parkinson's; stroke; chronic kidney disease; Crohn's; diabetes mellitus type 1 and type 2; rheumatoid arthritis, osteoarthritis, psoriatic arthritis, lupus, gout, ulcerative colitis; acne; eczema; psoriasis; tendinitis; atherosclerosis; obesity; cancer, allergies; depression, among others.

The term “coadministration” or “combination therapy” is used to describe a therapy in which at least two active compounds in effective amounts are used to treat cancer or another disease state or condition as otherwise described herein at the same time. Although the term coadministration preferably includes the administration of two active compounds to the patient at the same time, it is not necessary that the compounds be administered to the patient at the same time, although effective amounts of the individual compounds will be present in the patient at the same time to effect an intended result. In cancer aspects of the invention, one or more compounds according to the present invention may be administered with one or more anti-cancer agents, including antimetabolites, alkylating agents, topoisomerase I and topoisomerase II inhibitors as well as microtubule inhibitors, among others. Anticancer compounds for use in the present invention include those described herein below, and mixtures thereof, among others. Preferred anticancer agents for use in the present invention in coadministration with one or more of the compounds disclosed herein include for example, fluorouracil, imiquimod, vismodegib, aldesleukin, dacarbazine, ipilimumab, vemurafenib or mixtures thereof.

Coadministration of one of the present compounds with another anticancer agent as otherwise described herein will often result in a synergistic enhancement of the anticancer activity of the other anticancer agent, an unexpected result. One or more of the present compounds may also be coadministered with another bioactive agent which treat the diseases or conditions as otherwise described herein), depending upon the desired therapeutic outcome and the disease state or condition treated.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention, inhibition or delay in the onset or progression of the disease, etc.

Treatment, as used herein, encompasses both prophylactic (reducing the likelihood of the occurrence of the disease) and therapeutic treatment, depending on the context of use. Compounds according to the invention can, for example, be administered prophylactically to a mammal in advance of the occurrence of disease. Prophylactic administration is effective to decrease the likelihood of the subsequent occurrence of disease in the mammal or decrease the severity of disease that has not yet occurred but that subsequently occurs. Alternatively, compounds according to the invention can, for example, be administered therapeutically to a mammal that is already afflicted by disease. In one embodiment of therapeutic administration, administration of the compounds according to the invention is effective to eliminate the disease; in another embodiment, administration of the compounds of the present invention is effective to inhibit the disease state or condition treated, including decrease the severity of the disease or lengthen the lifespan of the mammal so afflicted.

“Pharmaceutically acceptable” as used herein means that the compound or composition is suitable for administration to a subject to achieve the treatments described herein, without unduly deleterious side effects in light of the severity of the disease and necessity of the treatment.

“Inhibit” as used herein refers to the partial or complete elimination of a potential effect, while inhibitors are compounds that have the ability to inhibit.

The present invention includes the compositions comprising the pharmaceutically acceptable salt. i.e., the acid or base addition salts of compounds of the present invention and their derivatives, where applicable. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

The term “cancer” shall refer to a proliferation of tumor cells having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. The term cancer also within context, includes drug resistant cancers, including multiple drug resistant cancers. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, thyroid, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas, among others.

The term “additional anti-cancer agent” is used to describe an additional compound which may be coadministered with one or more compounds of the present invention in the treatment of cancer. Such agents include, for example, antimetabolites, alkylating agents, topoisomerase I and topoisomerase II inhibitors as well as microtubule inhibitors, among others. Specific anti-cancer agents for use in the present invention include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimunmab, gossypol, Bio 111, 131-1-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gerncitabine, doxorubicin, irinotecan, liposomal doxorubicin, 5′-deoxy-5-fluorouridine, vincristine, temozolomide, ZK-304709, seliciclib PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHLR-258,); 3-15-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac (imitinib), hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, rahitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SUS416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210. LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others, including for example immunotherapies, such as ipilimumab, pembrolizumab, nivolumab, alemtuzumab, brentuximab vedotin, blinatumomab and cetuximab, among others.

Because of the dual activity exhibited by compounds according to the present invention, these compounds may be used to treat numerous disease states or conditions in patients or subjects who suffer from those conditions or disease states or are at risk for those conditions. In this method at least one compound, alone or in further combination with at least one additional bioactive agent in an effective amount is administered to a patient in need of therapy to treat or reduce the likelihood of the occurrence of the condition(s) or disease state(s). The compounds and methods of the invention are useful for treating or reducing the likelihood of any the following diseases: These disease states and/or conditions include for example, Alzheimer's; ALS; autism; bipolar disorder; brain injury; chronic pain; chronic inflammatory demyelinating polyneuropathy (CIPD); diabetic neuropathy; epilepsy; fibromyalgia; Huntington's; inflammatory myopathy; meningitis; migraine; multiple sclerosis (MS); Parkinson's; stroke; chronic kidney disease; Crohn's; diabetes mellitus type 1 and type 2; rheumatoid arthritis; osteoarthritis; psoriatic arthritis; lupus; gout; ulcerative colitis; acne; eczema; psoriasis; tendinitis; atherosclerosis; obesity; cancer; allergies; depression, among others. Compounds also are useful in treating skin diseases and/or conditions including, for example, Acrodermatitis, Cellulite, Cryotherapy, Cutaneous skin tags, Dermatitis herpetiformis, Dry skin, Ectodermal dysplasia, Epidermolysis bullosa, Erythema multiforme, Erythema nodosum, Erythema toxicum, Granuloma annulare, Henoch-Schonlein purpura, Hyperelastic skin, lchthyosis vulgaris, Idiopathic or primary livedo reticularis, lntertrigo, Keratosis pilaris, Lamellar ichthyosis, Lichen planus, Lichen simplex chronicus, Milia, Nikolsky's sign, Perioral dermatitis, Pityriasis rosea, Pityriasis rubra pilaris. Polymorphic light eruption, Preauricular tag or pit, Purpura Pyogenic granuloma, Sebaceous cyst, Seborrheic dermatitis, Seborrheic keratosis, Skin and hair changes during pregnancy. Skin blushing/flushing, Skin discoloration—bluish, Skin graft, Skin lesion biopsy, Skin lumps, Skin turgor, Stasis dermatitis and ulcers, Striae. Subcutaneous emphysema, Vesicles, Wood's lamp examination, Xanthoma, Xeroderma pigmentosa, Xerosis, Eczema, Impetigo, Itching, Psoriasis, Rashes, Scleroderma. Skin Aging, Skin Cancer, Skin Infections and Skin Pigmentation Disorders, among others.

Compositions according to the present invention may be administered by any conventional means known in the art. Pharmaceutical formulations include those suitable for oral, rectal, nasal, topical (including buccal and sub-lingual), vaginal or parenteral (including intramuscular, sub-cutaneous and intravenous) administration, but compositions which are administered by topical and/or transdermal route of administration directly at the site in the skin of the disease state or condition to be treated are preferred. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous, intramuscular or intraperitoneal dosage form, as well as via other parenteral routes, such as transdermal, buccal, subcutaneous, suppository or other route, including via inhalation or intranasally. Topical routes of administration may be preferred when treating skin disease states and/or conditions. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymetbylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration, where applicable, can be prepared by mixing an active agent and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the active.

Dosage forms for topical administration include ointments, powders, sprays and inhalants. The compound(s) are admixed under sterile conditions with a physiologically acceptable carrier, and any preservatives, buffers, and/or propellants that may be required. Opthalmic formulations, eye ointments, powders, and solutions are also contemplated as being within the scope of this invention.

Generally, dosages and routes of administration of the pharmaceutical compositions and therapeutic compounds described herein are determined according to the size and condition of the subject, according to standard pharmaceutical practices. Dose levels employed can vary widely, and can readily be determined by those of skill in the art.

Typically, amounts in the milligram up to gram quantities are employed.

The dosage administered pursuant to the present invention is an effective amount for producing an intended result and will vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active compound can be about 0.01 to 500 milligrams per kilogram of body weight or more, often 0.1 milligrams to 250 milligrams per kilogram of body weight. Ordinarily, 0.5 to 50, and often 1 to 25 milligrams per kilogram per day given in divided doses 1 to 6 times a day or in sustained release form is effective to obtain desired results.

The active compounds may be used at a concentration of 0.01 to 99.9 weight percent of the formulation, or in some cases a concentration of 0.001 to 99.9 weight percent of the formulation. Preferably the pharmaceutical formulation is in unit dosage form. The unit dosage form can be a capsule or tablet itself, or the appropriate number of any of these. The quantity of active compound in a unit dose of composition may be varied or adjusted from about 0.05 to several grams, often 0.1 to about 1000 milligrams or more or about 1 milligram to 500) milligrams according to the particular treatment involved. Compositions (dosage forms) suitable for internal administration contain from about 1 milligram to about 1000 milligrams of active compound per unit. In these pharmaceutical compositions the active compound will ordinarily be present in an amount of about 0.5-95% by weight based on the total weight of the composition.

### Disease Treatment Using the Present Compounds

Treatment, as defined herein, is the amelioration of the symptoms associated with disease. Symptoms may be reduced either by decreasing the level of the disease itself, or by decreasing the symptoms associated with the disease. The subject of the treatment is preferably a mammal, such as a domesticated farm animal (e.g., cow, horse, pig, or a domesticated pet (e.g., dog, cat). More preferably, the subject is a human.

As noted herein, and without being bound by any particular theory, one mechanism by which administration of the compounds according to the present invention may treat disease is through inhibition of the activity of NF-κB and up-regulation (increasing) the activity of Nrf2. Inhibition of NF-κB results in a decrease in NF-κB activity, and includes direct inhibition and indirect inhibition. Direct inhibition is the direct effect of a compound on NF-κB and its activity. For example, one type of direct inhibition of NF-κB is a block of NF-κB DNA interactions. Indirect inhibition, on the other hand, involves the effect of a compound involved in the regulation of NF-κB that leads to a decrease in NF-κB activity. For example, as phosphorylation of the NF-κB regulator IκB by IκB kinases (IKK) or Src family kinases (SFK) results in a dysregulation of NF-κB, and an according increase in NF-κB activity, inhibition of IKK or SFK by the present compounds provides an example of indirect inhibition.

Increase in Nrf2 results in an increase or up-regulation of Nfr2 activity, and includes direct agonist activity and indirect agonist activity. Direct enhancement is the direct effect of a compound on Nrf2 (or its subunits) and its activity. Indirect inhibition, on the other hand, involves the effect of a compound according to the present invention in the regulation of Nrf2 that leads to an increase in Nrf2 activity.

### EXAMPLES

### Chemical Synthesis

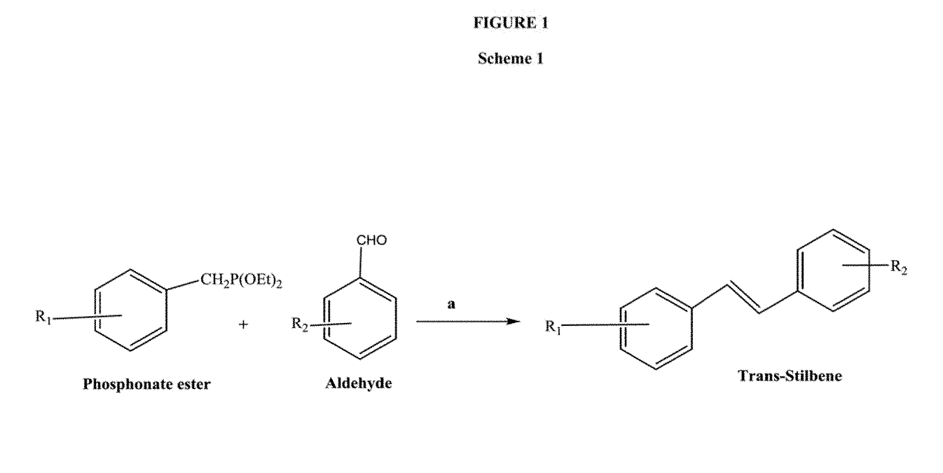

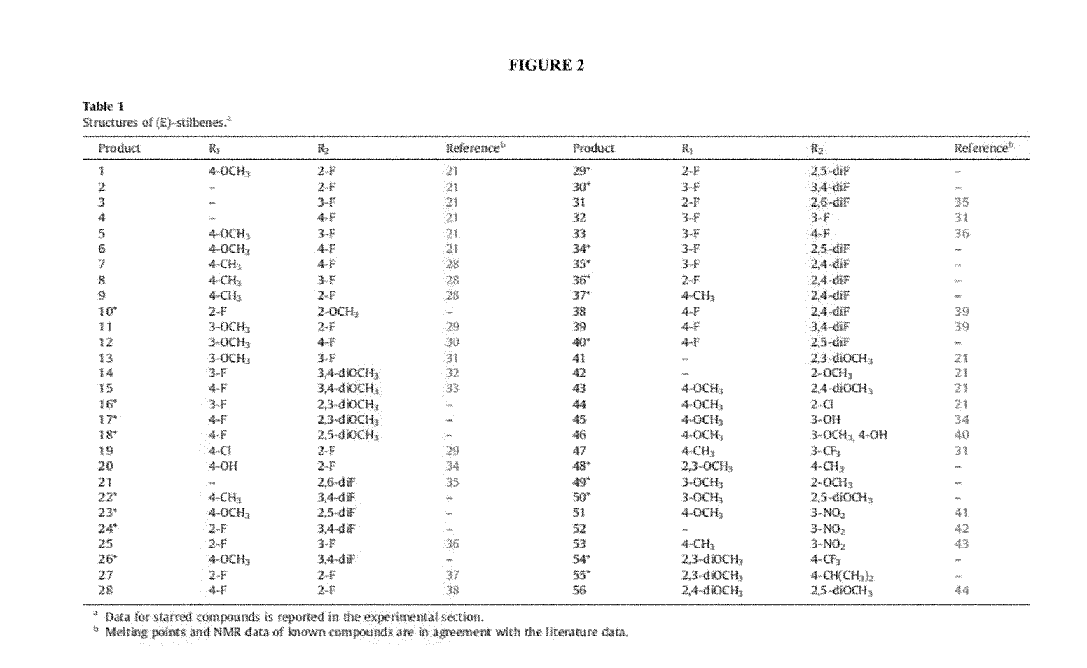

The synthesis of 56 substituted trans (E)-stilbenes (FIG. 2, Table 1) was accomplished using Horner-Wadsworth-Emmons (HWE) olefination chemistry.²⁴ The starred compounds in Table 1 are new to the literature, and their synthesis is described in FIG. 1, Scheme 1.²⁵ Synthesis of the other compounds in Table 1 was accomplished using a method reported previously.²¹ HWE chemistry was used to avoid formation of a mixture of E and Z isomers and formation of triphenylphosphine oxide, which complicates the purification process.

The required phosphonate ester starting materials were prepared in high yields by the classical solvent free Michaelis-Arbuzov reaction of substituted benzyl chlorides or bromides with triethylphosphite at 130° C.²⁶ Removal of excess triethylphosphite and chloroethane or bromoethane product was carried out by vacuum distillation. Further purification using a short silica gel column and eluting with ethyl acetate/hexane provided pure phosphonate esters as oils.²⁷

Reaction of the appropriately substituted diethyl benzylphosphonate ester with a corresponding freshly distilled substituted benzaldehyde in dry tetrahydrofuran using sodium hydride as the base (FIG. 1, Scheme 1) afforded the 20 new substituted stilbenes (FIG. 2, Table 1) exclusively in the E conformation. There was no detectable Z isomer, and the trans geometry of the substituted stilbenes was confirmed by the coupling constants of approximately 16.5 Hz for the olefinic protons in the proton NMR spectra. The reaction conditions allowed for a straightforward workup since the diethylphosphoric acid byproducts are water-soluble and were easily removed by extraction. Purification of the benzaldehydes and phosphonate esters was found to be critical for obtaining good yields and pure products.

In the case of enone compounds according to the present invention, these are synthesized by methods which are well-known in the art and which are alternatively presented in Deck, et al., Eur. J Med Chem. 2018 Jan. 1; 143:854-865, which is incorporated by reference herein.

### EXPERIMENTAL

### Reporter Assays:

A Nrf2-ARE reporter-HepG2 stable cell line (BPS Bioscience, San Diego, Calif.) is grown in a humidified atmosphere at 37° C. in 5% CO₂95% air. The cells are maintained in MEM medium with Earles balanced salts and L-glutamine supplemented with 10% fetal bovine serum (FBS), 1 mM sodium pyruvate, 1% non-essential amino acids, 100 units/ml penicillin, 100 μg/ml streptomycin, and 400 μg/ml Geneticin. One day prior to treatment, the Nrf2-ARE cells are plated into 24-well cell culture plates at approximately 30% confluency in the above media without Geneticin. The following day, fresh media with or without substituted trans stilbene or sulforaphane is applied to the cells. DMSO concentrations are kept at 0.1%. The cells are again placed in a humidified atmosphere at 37° C. in 5% CO₂/95% air for 5 hours. Plate wells are gently washed with phosphate buffered saline (PBS) pH 7.4 and lysed with 1× passive lysis buffer (Promega, Madison, Wis., USA). The subsequent lysates are analyzed with the Luciferase Assay System (Promega) utilizing a GloMax 20/20 luminometer (Promega, Sunnyvale, Calif., USA). The firefly luciferase relative light units are normalized to protein (mg/ml) with BCATM Protein Assay Kit protein (Pierce, Rockford, Ill., USA)

An NFκB reporter stable cell line from human 293Tembryonic kidney cells (293T/NFκB-luc) (Panomics, Inc., Redwood City, Calif.) was grown in a humidified atmosphere at 37° C. in 5% CO2/95% air. The cells were maintained in Dulbecco's modified Eagle's medium (DMEM-high glucose containing 4 mM glutamine) supplemented with 10% fetal bovine serum (FBS), 1 mM sodium pyruvate, 100 U/ml penicillin, 100 ug/ml streptomycin, and 100 ug/ml hygromycin (Gibco/Invitrogen, Carlsbad, Calif.) to maintain cell selection. One day prior to treatment, the 293T/NFκB-luc cells were plated into 24-well cell culture plates (Costar, Cambridge, Mass.) at approximately 70% confluency in the above media without hygromycin. The following day cells were fed fresh media 1 h prior to treatment. Media with or without recombinant tumor necrosis factor alpha (TNFa) (R&D Biosciences/Clontech, Palo Alto, Calif.) were then applied to the cells at 20 ng/ml followed by immediate treatments with inhibitor. The cells were placed again in a humidified atmosphere at 37° C. in 5% CO2/95% air for 7 h. Plate wells were gently washed with phosphate-buffered saline, pH 7.4, and lysed with 1× passive lysis buffer (Promega, Madison, Wis.). The subsequent lysates were analyzed with the Luciferase Assay System (Promega) utilizing a TD-20/20 luminometer (Turner Designs, Sunnyvale, Calif.). The firefly luciferase relative light units were normalized to protein (mg/ml) with BCATM Protein Assay Kit (Pierce, Rockford, Ill.) and standardized to percent of control (TNFa control). For assays of cell viability, cells were treated similarly as above and with 15 uM inhibitor. Alter washing, cells were treated with 100 ul media and 20 ul CellTiter 96 AQueous One Solution reagent for 1 h and then read at 490 nm with a Spectromax plate reader.

### Synthesis

Reagents were purchased from commercial sources (Aldrich, Acros, etc.). Tetrahydrofuran was distilled from lithium aluminum hydride. Thin layer chromatography was carried out on silica gel 60F254 plates. All compounds were shown to be >98% pure by ¹H NMR and/or ¹³C NMR unless otherwise noted. Column chromatographic separations were performed by using EM type 60 silica gel (230-400 mesh). Melting points were taken on a Thomas-Hoover Uni-Melt capillary melting point apparatus and reported uncorrected. Unless otherwise noted, ¹H spectra were recorded by using CDCl₃ solutions at 300 MHz; ¹³C NMR spectra were recorded in CDCl₃ at 75 MHz; ¹⁹F were recorded in CDCl₃ at 282 MHz. Chemical shifts are reported in ppm relative to CDCl₃ at 7.24 ppm for ¹H NMR and 77.0 ppm for ¹³C NMR and the external standard hexafluorobenzene for 1′NMR at −164.9 ppm. Peak assignments were made with the aid of DEPT spectra. ¹H NMR data are reported as follows: chemical shift, multiplicity (s=singlet, d=doublet, t=triplet, q=quartet, dd=doublet of doublets, td=triplet of doublets, m=multiplet), coupling constant (J in Hz) and integration. High resolution mass spectra (HRMS) were obtained at the UNM Mass Spectrometry Facility, Albuquerque, N. Mex.

### General Procedure for Synthesis of Phosphonate Esters

Benzyl chloride or benzyl bromide derivatives (1 eq) were added to triethylphosphite (1.5 eq) and heated to 130° C. for 20 h. After cooling, the resulting crude product was distilled in vacuo to remove excess triethylphosphite and ethyl chloride or ethyl bromide. Purification by filtration through a pad of silica gel (70% ethyl acetate/30% hexanes) gave the phosphonate ester products as colorless oils.

### General Procedure for Synthesis of Stilbenes

The appropriately substituted phosphonate ester (10 mmol) was dissolved in dry tetrahydrofuran (20 ml) and stirred at 0-5° C. Sodium hydride (25 mmol) was added to the solution slowly and after thirty minutes the appropriate freshly distilled aldehyde (10 mmol) in tetrahydrofuran (30 ml) was added dropwise. The mixture was allowed to stir at room temperature overnight. In order to increase the yield, compounds 35, 37 and 40 were heated under reflux for 3-4 hours. The mixture was cooled and quenched with ice water (10 ml) and poured onto ice. Dilute hydrochloric acid (1M) was added until acidic and the solution was extracted with ethyl acetate (4×50 ml). The combined organic layers were washed with saturated salt and dried over magnesium sulfate. Filtration and evaporation of the ethyl acetate afforded crude stilbene products as oils or solids. The solids were crystallized from 95% ethanol to afford crystalline stilbenes. The oils were chromatographed on silica gel using methylene chloride to give pure products.

### (E)-1-Fluoro-2-(2-methoxystyryl)benzene 10

98% yield, white crystals; mp 34-35° C.; ¹H NMR (CDCl₃, 300 MHz): δ7.66 (dt, J=7.8, 1.6 Hz, 1H), 7.62 (dd, J=7.6, 1.5 Hz, 1H), 7.54 (d, J=16.6 Hz, 1H), 7.27 (d, J=16.6 Hz, 1H), 7.22 (m, 2H), 7.13 (dt, J=7.5, 1.3 Hz, 1H), 7.05 (dt, J=8.0, 1.3 Hz, 1H), 6.98 (t, J=7.4 Hz, 1H), 6.91 (d, J=8.3 Hz, 1H), 3.88 (s, 3H). ¹³C NMR (CDCl₃, 75 MHz): δ 160.5 (d, J=247.7 Hz), 157.2 (s), 129.2 (s), 128.6 (d, J=8.4 Hz), 127.0 (d, J=3.4 Hz), 126.7 (s), 126.5 (s), 126.0 (d, J=12.1 Hz), 125.7 (d, J=3.6 Hz), 124.2 (d, J=3.1 Hz), 121.1 (d, J=4.0 Hz), 120.9 (s), 115.5 (d, J=22.2 Hz), 110.8 (s), 55.7 (s). ¹⁹F (CDCl₃, 282 MHz): δ −116.9 (s, 1F). HRMS (EI) calcd for C₁₅H₁₃FO [M]⁺: 228.0950; found, 228.0950.

### (E)-1-(3-Fluorostyryl)-2,3-dimethoxybenzene 16

98% yield oil; ¹H NMR (CDCl₃, 300 MHz):

δ 7.45 (d, J=16.5 Hz, 1H), 7.27 (m, 3H), 7.23 (dd, J=7.9, 1.3 Hz, 1H), 7.08 (d, J=16.5 Hz, 1H), 7.06 (t, J=8.1 Hz, 1H), 6.96 (m, 1H), 6.86 (dd, J=8.1, 1.4 Hz, 1H), 3.88 (s, 3H), 3.87 (s, 3H). ¹³C NMR (CDCl₃, 75 MHz): δ 163.2 (d, J=245.1 Hz), 153.3 (s), 147.3 (s), 140.1 (d, J=7.6 Hz)), 131.0 (s), 130.1 (d, J=8.4 Hz), 128.7 (s), 124.3 (s), 124.2 (s), 122.6 (s), 117.9 (s), 114.4 (d, J=21.5 Hz), 112.9 (d, J=21.8 Hz), 111.8 (s), 61.2 (s), 56.0 (s). ¹⁹F (CDCl₃, 282 MHz): δ −111.9 (s, 1F). HRMS (EI) calcd for C₁₆H₁₅FO₂ [M]⁺: 258.1056; found, 258.1058.

### (E)-1-(4-Fluorostyryl)-2,3-dimethoxybenzene 17

87% yield, white crystals; mp 50-51° C.; ¹H NMR (CDCl₃, 300 MHz): δ 7.50 (dd, J=8.4, 5.6 Hz, 2H), 7.36 (d, J=16.5 Hz, 1H), 7.21 (d, J=7.8 Hz, 1H), 7.07 (d, J=16.4 Hz, 1H), 7.04 (m, 3H), 6.83 (d, J=7.6 Hz, 1H), 3.87 (s, 3H), 3.85 (s, 3H). ¹³C NMR (CDCl₃, 75 MHz): δ 162.4 (d, J=247.1 Hz), 153.2 (s), 147.0 (s), 133.9 (d, 2.9 Hz), 131.4 (s), 128.7 (s), 128.2 (d, J=7.9 Hz), 124.2 (s), 122.8 (s), 117.8 (s), 115.6 (d, J=21.6 Hz), 111.5 (s), 61.1 (s), 55.8 (s). ¹⁹F (CDCl₃, 282 MHz): δ −112.7 (s, 1F). HRMS (EI) calcd for C₁₆H₁₅FO₂ [M]⁺: 258.1056; found, 258.1060.

### (E)-2-(4-Fluorostyryl)-1,4-dimethoxybenzene 18

74% yield, white crystals; mp 58-59° C.; ¹H NMR (CDCl₃, 300 MHz): δ 7.49 (dd, J=8.7, 5.5 Hz, 2H), 7.37 (d, J=16.4 Hz, 1H), 7.13 (d, J=2.6 Hz, 1H), 7.05 (d, J=16.4 Hz, 1H), 7.03 (t, J=8.7 Hz, 2H), 6.80 (m, 2H), 3.83 (s, 3H), 3.81 (s, 3H). ¹³C NMR (CDCl₃, 75 MHz): δ 162.3 (d, J=247.0 Hz), 153.8 (s), 151.4 (s), 134.0 (d, 3.1 Hz), 128.1 (s), 128.0 (d, J=7.7 Hz), 123.1 (s), 115.6 (d, J=21.6 Hz), 113.7 (s), 112.2 (s), 111.7 (s), 56.2 (s), 55.7 (s). ¹⁹F (CDCl₃, 282 MHz): δ −112.9 (s, 1F). HRMS (EI) calcd for C₁₆H₃FO₂ [M]⁺: 258.1056; found, 258.1057.

### (E)-1,2-Difluoro-4-(4-methylstyryl)benzene 22

97% yield, white crystals; mp 90-92° C.; ¹H NMR (CDCl₃, 300 MHz): δ 7.38 (d, J=8.1 Hz, 2H), 7.29 (m, 1H), 7.16 (d, J=8.1 Hz, 2H), 7.13 (m, 2H), 6.99 (d, J=16.4 Hz, 1H), 6.92 (d, J=16.4 Hz, 1H), 2.36 (s, 3H). ¹³C NMR (CDCl₃, 75 MHz): δ 151.7 (dd, J=69.7, 13.3 Hz), 148.5 (dd, J=70.7, 13.3 Hz), 138.0 (s), 134.9 (t, J=5.0 Hz), 133.9 (s), 129.7 (s), 129.5 (s), 126.5 (s), 125.5 (s), 122.6 (dd, J=6.1, 2.9 Hz), 117.3 (d, J=17.5 Hz), 114.5 (d, J=17.6 Hz), 21.3 (s). ¹⁹F (CDCl₃, 282 MHz): δ −136.3 (d, J=20.9 Hz, 1F), −137.7 (d, J=20.9 Hz, 1F). HRMS (EI) calcd for C₁₅H₂F₂ [M]⁺: 230.0907; found, 230.0911.

### (E)-1,4-Difluoro-2-(4-methoxystyryl)benzene 23

60% yield, pale yellow crystals; mp 109-110° C.; ¹H NMR (CDCl₃, 300 MHz): δ 7.46 (d, J=8.6 Hz, 2H), 7.25 (m, 1H), 7.06 (s, 2H), 6.98 (m, 1H), 6.89 (d, J=8.6 Hz, 2H), 6.86 (m, 1H), 3.81 (s, 3H). ¹³C NMR (CDCl₃, 75 MHz): δ 159.8 (s), 159.2 (d, J=200.2 Hz), 156.0 (d, J=201.6 Hz), 131.5 (d, J=3.9 Hz), 129.5 (s), 128.1 (s), 127.0 (dd, J=14.5, 8.2 Hz), 117.6 (s), 116.7 (dd, J=25.4, 8.7 Hz), 114.5 (dd, J=24.5, 9.0 Hz), 114.2 (s), 112.4 (dd, J=24.6, 3.8 Hz), 55.3 (s). ¹⁹F (CDCl₃, 282 MHz): δ −117.7 (d, J=17.2 Hz, 1F), −122.9 (d, J=17.2 Hz, 1F). HRMS (EI) calcd for C₁₅H₁₂F₂O [M]⁺: 246.0856; found, 246.0852.

### (E)-1,2-Difluoro-4-(2-fluorostyryl)benzene 24

82% yield, white crystals; mp 85-86° C. ¹H NMR (CDCl₃, 300 MHz): δ 7.56 (dt, J=7.7, 1.6 Hz, 1H), 7.33 (dt, J=7.7, 1.8 Hz, 1H), 7.23 (m, 2H), 7.17 (d, J=16.4 Hz, 1H), 7.10 (m, 3H), 7.05 (d, J=16.3 Hz, 1H). ¹³C NMR (CDCl₃, 75 MHz): δ 160.7 (d, J=247.9 Hz), 152.1 (dd, J=44.2, 12.0 Hz), 148.8 (dd, J=46.6, 12.0 Hz), 134.7 (t, J=6.2 Hz), 129.4 (d, J=8.4 Hz), 128.9 (s), 127.3 (d, J=3.2 Hz), 124.7 (d, J=11.9 Hz), 124.4 (d, J=2.7 Hz), 123.1 (dd, J=5.3, 3.0 Hz), 122.3 (s), 117.6 (d, J=17.5 Hz), 116.1 (d, J=22.1 Hz), 115.0 (d, J=17.6 Hz). ¹⁹F (CDCl₃, 282 MHz): δ −116.0 (s, 1F), −136.0 (d, J=20.8 Hz, 1F), −136.8 (d, J=21.1 Hz, 1F). HRMS (EI) calcd for C₁₄H₉F₃ [M]⁺: 234.0656; found, 234.0652.

### (E)-1,2-Difluoro-4-(4-methoxystyryl)benzene 26

66% yield, white crystals; mp 73-74° C.; ¹H NMR (CDCl₃, 300 MHz): δ 7.41 (d, J=8.6 Hz, 2H), 7.27 (m, 1H), 7.11 (m, 2H), 6.94 (d, J=16.5 Hz, 1H), 6.88 (d, J=8.7 Hz, 2H), 6.83 (d, J=16.5 Hz, 1H), 3.81 (s, 3H). ¹³C NMR (CDCl₃, 75 MHz): δ 159.8 (s), 151.8 (dd, J=79.6, 12.7 Hz), 148.6 (dd, J=81.0, 13.2 Hz), 135.2 (t, J=4.9 Hz), 129.6 (d, J=13.0 Hz), 129.5 (s), 128.0 (s), 124.5 (s), 122.5 (dd, J=5.4, 2.8 Hz), 117.5 (d, J=17.4 Hz), 114.5 (d, J=16.0 Hz), 114.4 (s), 55.5 (s). ¹⁹F (CDCl₃, 282 MHz): δ −136.4 (d, J=20.9 Hz, 1F), −138.1 (d, J=20.8 Hz, 1F). HRMS (EI) calcd for C₁₅H₁₂F₂O [M]⁺: 246.0856; found, 246.0860.

### (E)-1,4-Difluoro-2-(2-fluorostyryl benzene 29

88% yield, white crystals; mp 79-81° C.; ¹H NMR (CDCl₃, 300 MHz): δ 7.60 (dt, J=7.7, 1.6 Hz, 1H), 7.27 (s, 2H), 7.26 (m, 2H), 7.13 (dd, J=7.6, 1.1 Hz, 1H), 7.08 (m, 1H), 7.00 (m, 1H), 6.90 (m, 1H). ¹³C NMR (CDCl₃, 75 MHz): δ 160.6 (d, J=250.4 Hz), 159.3 (d, J=185.2 Hz), 156.1 (d, J=189.0 Hz), 129.6 (d, J=8.5 Hz), 127.2 (d, J=2.9 Hz), 126.5 (dd, J=14.4, 7.9 Hz), 124.6 (d, J=12.0 Hz), 124.3 (d, J=3.3 Hz), 124.1 (s), 122.0 (s), 116.8 (dd, J=25.3, 8.9 Hz), 115.9 (d, J=22.2 Hz), 115.5 (dd, J=24.5, 8.8 Hz), 112.8 (dd, J=24.7, 3.5). ¹⁹F (CDCl₃, 282 MHz): δ −115.9 (s, 1F), −117.3 (d, J=17.2 Hz, 1F), −122.4 (d, J=17.2 Hz, 1F). HRMS (EI) calcd for C₁₄H₉F₃ [M]⁺: 234.0656; found, 234.0654.

### (E)-1,2-Difluoro-4-(3-fluorostyryl)benzene 30

95% yield, white crystals; mp 48-49° C., ¹H NMR (CDCl₃, 300 MHz): δ 7.30 (m, 211), 7.16 (m, 4H), 6.98 (m, 111), 6.96 (d, J=16.5 Hz, 1H), 6.90 (d, J=16.5 Hz, 1H). ¹³C NMR (CDCl₃, 75 MHz): δ 163.2 (d, J=245.6 Hz), 152.0 (dd, J=42.1, 11.1 Hz), 148.7 (dd, J=44.7, 11.1 Hz), 139.1, (d, J=7.8 Hz), 134.2 (t, J=5.9 Hz), 130.2 (d, J=8.4 Hz), 128.5 (s), 127.8 (s), 122.9 (dd, J=6.2, 3.6 Hz), 122.6 (d, J=2.3 Hz), 117.5 (d, J=17.5 Hz), 114.9 (d, J=5.3 Hz), 114.7 (s), 112.8 (d, J=21.9 Hz). ¹⁹F (CDCl₃, 282 MHz): δ −111.4 (s, 1F), −135.8 (d, J=20.9 Hz, 1F), −136.5 (d, J=20.8 Hz, 1F). HRMS (EI) calcd for C₄H₉F₃ [M]⁺: 234.0656; found, 234.0660.

### (E)-1,4-Difluoro-2-(3-fluorostyryl)benzene 34

89% yield, white crystals; mp 73-74° C.; ¹H NMR (CDCl₃, 300 MHz): δ 7.27 (m, 4H), 7.18 (d, J=16.5 Hz, 1H), 7.06 (d, J=16.6 Hz, 1H), 7.01 (m, 2H), 6.91 (m, 1H). ¹³C NMR (CDCl₃, 75 MHz): δ 163.2 (d, J=245.7 Hz), 159.3 (d, J=179.4 Hz), 156.0 (d, J=183.7 Hz), 139.0 (d, J=7.7 Hz), 130.8 (s), 130.2 (d, J=8.3 Hz), 126.1 (m), 122.8 (d, J=2.0 Hz), 121.2 (s), 116.9 (dd, J=25.3, 8.8 Hz), 115.5 (dd, J=24.5, 8.7 Hz), 115.1 (d, J=21.6 Hz), 113.1 (d, J=21.9 Hz), 112.8 (dd, J=24.6, 3.8 Hz). ¹⁹F (CDCl₃, 282 MHz): δ −111.5 (s, 1F), −117.3 (d, J=17.2 Hz, 1F), −122.0 (d, J=17.2 Hz, 1F). HRMS (EI) calcd for C₁₄H₉F₃ [M]⁺: 234.0656; found, 234.0659.

### (E)-2,4-Difluoro-1-(3-fluorostyryl)benzene 35

67% yield, white crystals; mp 60-61° C.; ¹H NMR (CDCl₃, 300 MHz): δ 7.50 (dd, J=15.0, 8.3 Hz, 1H), 7.23 (m, 3H), 7.16 (d, J=16.5 Hz, 1H), 7.04 (d, J=16.5 Hz, 1H), 6.88 (m, 3H). ¹³C NMR (CDCl₃, 75 MHz): δ 163.2 (d, J=245.5 Hz), 163.1 (dd, J=143.9, 12.3 Hz), 159.8 (dd, J=146.1, 12.2), 139.5 (d, J=7.6 Hz), 130.2 (d, J=8.4 Hz), 129.4 (s), 128.0 (dd, J=9.4, 5.0 Hz), 122.5 (d, J=2.2 Hz), 121.3 (s), 121.1 (d, J=3.5 Hz), 114.8 (d, J=21.4 Hz), 112.9 (d, J=21.9 Hz), 11.7 (dd, J=22.0, 3.7 Hz), 104.2 (t, J=25.8 Hz). ¹⁹F (CDCl₃, 282 MHz): δ −108.6 (d, J=6.1 Hz, 1F), −111.6 (s, 1F), −111.7 (d, J=6.3 Hz, 1F). HRMS (EI) calcd for C₁₄H₉F₃ [M]⁺: 234.0656; found, 234.0658.

### (E)-2,4-Difluoro-1-(2-fluorostyryl)benzene 36

70% yield, white crystals; mp 84-85° C.; ¹H NMR (CDCl₃, 300 MHz): δ 7.60 (t, J=8.8 Hz, 111), 7.58 (t, J=8.7 Hz, 10H), 7.25 (s, 20), 7.22 (m, 1H), 7.13 (dt, J=7.6, 1.3 Hz, 1H), 7.06 (dt, J=9.5, 1.3 Hz, 1H), 6.83 (m, 2H). ¹³C NMR (CDCl₃, 75 MHz): δ 162.6 (dd, J=250.5, 13.9 Hz), 162.3 (t, J=5.1 Hz), 158.9 (t, J=10.2 Hz), 129.3 (d, J=8.4 Hz), 128.1 (dd, J=9.5, 5.2 Hz), 127.2 (d, J=3.1 Hz), 125.1 (d, J=11.9 Hz), 124.4 (d, J=3.2 Hz), 122.9 (s) 122.2 (s), 121.7 (dd, J=11.7, 3.6 Hz), 116.0 (d, J=22.2 Hz), 111.8 (dd, J=21.6, 3.5 Hz), 104.3 (t, J=25.7 Hz). ¹⁹F (CDCl₃, 282 MHz): δ −108.8 (s, 1F), −112.2 (d, J=5.6 Hz, 1F), −116.4 (s, 1F). HRMS (EI) calcd for C₁₄H₉F₃ [M]⁺: 234.0656; found, 234.0658.

### (E)-2,4-Difluoro-1-(4-methylstyryl)benzene 37

32% yield, white crystals; mp 72-73° C.; ¹H NMR (CDCl₃, 300 MHz): δ 7.55 (dd, J=15.0, 8.4 Hz, 1H), 7.89 (d, J=7.9 Hz, 2H), 7.47 (d, J=7.5 Hz, 2H), 7.16 (d, J=16.5 Hz, 1H), 7.07 (d, J=16.5 Hz, 1H), 6.85 (m, 2H), 2.35 (s, 3H). ¹³C NMR (CDCl₃, 75 MHz): δ 162.9 (dd, J=131.0, 12.1 Hz), 159.5 (dd, J=133.5, 12.0 Hz), 137.9 (s), 134.3 (s), 130.5 (s), 129.5 (s), 127.7 (dd, J=9.2, 5.4 Hz), 126.5 (s), 121.8 (dd, J=12.5, 3.8 Hz), 118.9 (s), 111.5 (dd, J=21.5, 2.8 Hz), 104.1 (t, J=25.7 Hz), 21.4 (s). ¹⁹F (CDCl₃, 282 MHz): δ −109.7 (d, J=5.3 Hz, 1F), −112.4 (d, J=4.4 Hz, 1F). HRMS (E) calcd for C₁₅H₁₂F₂[M]⁺: 230.0907; found, 230.0912.

### (E)-1,4-Difluoro-2-(4-fluorostyryl)benzene 40

50% yield, buff crystals; mp 76-77° C.; ¹H NMR (CDCl₃, 300 MHz): δ 7.48 (dd, J=8.5, 5.5 Hz, 2H), 7.25 (m, 1H), 7.09 (s, 2H), 7.00 (m, 3H), 6.90 (m, 1H). ¹³C NMR (CDCl₃, 75 MHz): δ 162.8 (d, J=248.3 Hz), 159.2 (d, J=190.6 Hz), 156.0 (d, J=192.8 Hz), 132.9 (d, J=3.0 Hz), 130.7 (d, J=4.3 Hz), 128.3 (d, J=8.0 Hz), 126.5 (m), 119.7 (s), 116.8 (dd, J=25.3, 8.9 Hz), 115.7 (d, J=21.8 Hz), 115.1 (dd, J=24.6, 8.9 Hz), 112.7 (dd, J=24.7, 3.0 Hz). ¹⁹F (CDCl₃, 282 MHz): δ −111.5 (s, 1F), −117.4 (d, J=17.0 Hz, 1F), −122.5 (d, J=16.9 Hz, 1F). HRMS (EI) calcd for C₄H₉F₃ [M]⁺: 234.0656; found, 234.0659.

### (E)-1,2-Dimethoxy-3-(4-methylstyryl)benzene 48

92% yield, white crystals; mp 38-40° C.; ¹H NMR (CDCl₃, 300 MHz): δ 7.43 (d, J=7.7 Hz, 2H), 7.41 (d, J=16.7 Hz, 1H), 7.22 (d, J=7.8 Hz, 1H), 7.14 (d, J=7.9 Hz, 2H), 7.08 (d, J=16.6 Hz, 1H), 7.02 (t, J=8.2 Hz, 1H), 6.79 (d, J=7.3 Hz, 1H), 3.83 (s, 6H), 2.34 (s, 3H). ¹³C NMR (CDCl₃, 75 MHz): δ 153.1, 146.8, 137.5, 134.9, 131.7, 129.8, 129.3, 126.6, 124.1, 121.9, 117.8, 111.1, 61.0, 55.7, 21.2. HRMS (EI) calcd for C₁₇H₁₈O₃ [M]⁺: 270.1256; found, 270.1258.

### (E)-1-Methoxy-2-(3-methoxystyryl)benzene 49

86% yield, white crystals; mp 49-50° C.; ¹H NMR (CDCl₃, 300 MHz): δ 7.55 (d, J=7.6 Hz, 1H), 7.48 (d, J=16.5 Hz, 1H), 7.23 (t. J=7.7 Hz, 1H), 7.18 (t, J=7.4 Hz, 1H), 7.09 (d, J=8.9 Hz, 1H), 7.06 (d, J=16.6 Hz, 1H), 7.05 (s, 1H), 6.92 (t, J=7.4 Hz, 1H), 6.83 (d, J=8.2 Hz, 1H), 6.76 (dd, J=8.0, 1.9 Hz, 1H), 3.80 (s, 3H), 3.76 (s, 3H). ¹³C NMR (CDCl₃, 75 MHz): δ 159.8, 156.9, 139.4, 129.5, 128.9, 128.7, 126.4, 126.2, 123.8, 120.7, 119.3, 113.0, 111.7, 110.9, 55.4, 55.1. HRMS (EI) calcd for C₁₆H₁₆O₂ [M]⁺: 240.1150; found, 240.1156.

### (E)-1,4-Dimethoxy-2-(3-methoxystyryl)benzene 50

96% yield, white crystals; mp 48-49° C.; ¹H NMR (CDCl₃, 300 MHz): δ 7.44 (d, J=16.4 Hz, 1H), 7.24 (t, J=7.9 Hz, 1H), 7.10 (m, 3H), 7.05 (d, J=16.4 Hz, 1H), 6.78 (m, 3H), 3.81 (s, 3H), 3.80 (s, 3H), 3.78 (s, 3H). ¹³C NMR (CDCl₃, 75 MHz): δ 159.9, 153.8, 151.5, 139.3, 129.5, 129.2, 127.1, 123.6, 119.4, 113.8, 113.2, 112.3, 111.8, 111.7, 56.2, 55.7, 55.2. HRMS (EI) calcd for C₁₇H₁₈O₃ [M]⁺: 270.1256; found, 270.1258.

### (E)-1,2-Dimethoxy-3-(4-(trifluoromethyl)styryl)benzene 54

84% yield, white crystals; mp 67-69° C.; ¹H NMR (CDCl₃, 300 MHz): δ 7.62 (d, J=9.3 Hz, 2H), 7.59 (d, J=9.7 Hz, 2H), 7.54 (d, J=16.7 Hz, 1H), 7.24 (d, J=7.8 Hz, 1H), 7.13 (d, J=16.6 Hz, 1H), 7.07 (t, J=8.0 Hz, 1H), 6.87 (d, J=8.0 Hz, 1H), 3.88 (s, 3H), 3.87 (s, 3H). ¹³C NMR (CDCl₃, 75 MHz): δ 153.1, 147.3, 141.2, 130.9, 128.3, 126.7, 125.6, 125.5, 124.2, 118.0, 112.0, 61.3, 56.0. ¹⁹F (CDCl₃, 282 MHz): δ −60.9 (s, 3F). HRMS (EI) calcd for C₁₇H₁₅F₃O₂[M]⁺: 308.3002; found, 308.3000.

### (E)-1-(4-Isopropylstyryl)-2,3-dimethoxybenzene 55

96% yield, oil; ¹H NMR (CDCl₃, 300 MHz): δ 7.35 (d, J=8.2 Hz, 2H), 7.26 (d, J=16.5 Hz, 1H), 7.24 (dd, J=6.3, 1.6 Hz, 1H), 7.17 (d, J=8.1 Hz, 2H), 7.14 (d, J=16.5 Hz, 1H), 7.07 (t, J=8.0 Hz, 1H), 6.84 (dd, J=8.1, 1.2 Hz, 1H), 3.89 (s, 3H), 3.87 (s, 3H), 2.94 (sept, J=6.9 Hz, 1H), 1.30 (d, J=6.9 Hz, 6H). ¹³C NMR (CDCl₃, 75 MHz): δ 153.1, 148.5, 146.8, 135.3, 131.8, 129.8, 126.7, 126.6, 124.1, 122.0, 117.8, 111.1, 61.0, 55.7, 33.9, 23.9. HRMS (EI) calcd for C₁₉H2₂O₂ [M]⁺: 282.1620; found, 282.1618.

### Results and Discussion

### Activation of Nrf2 by Resveratrol, LD55 and Sulforaphane

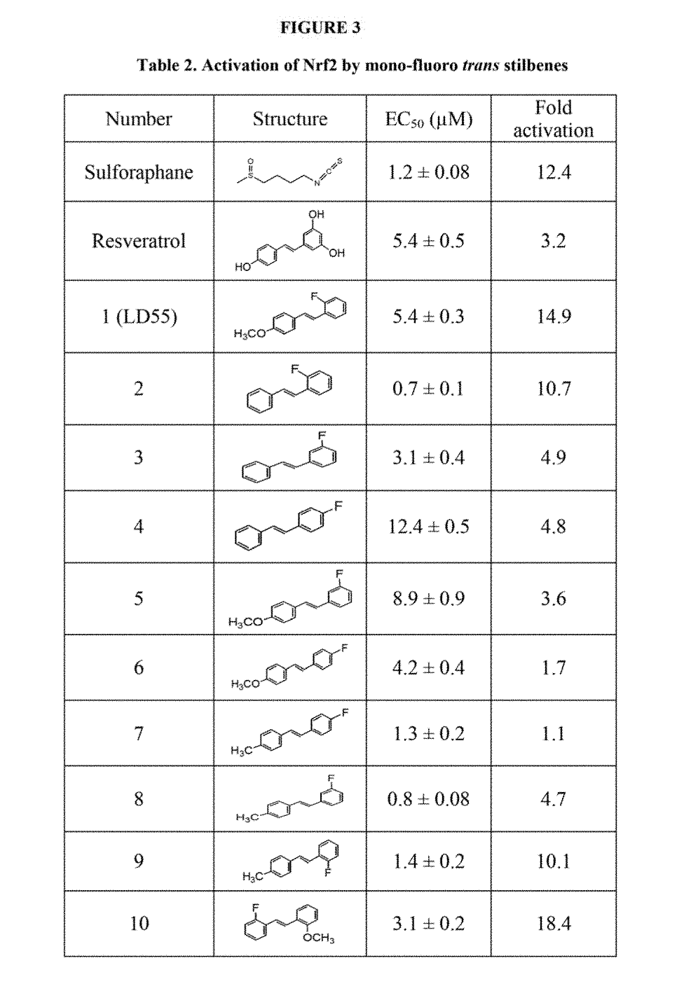

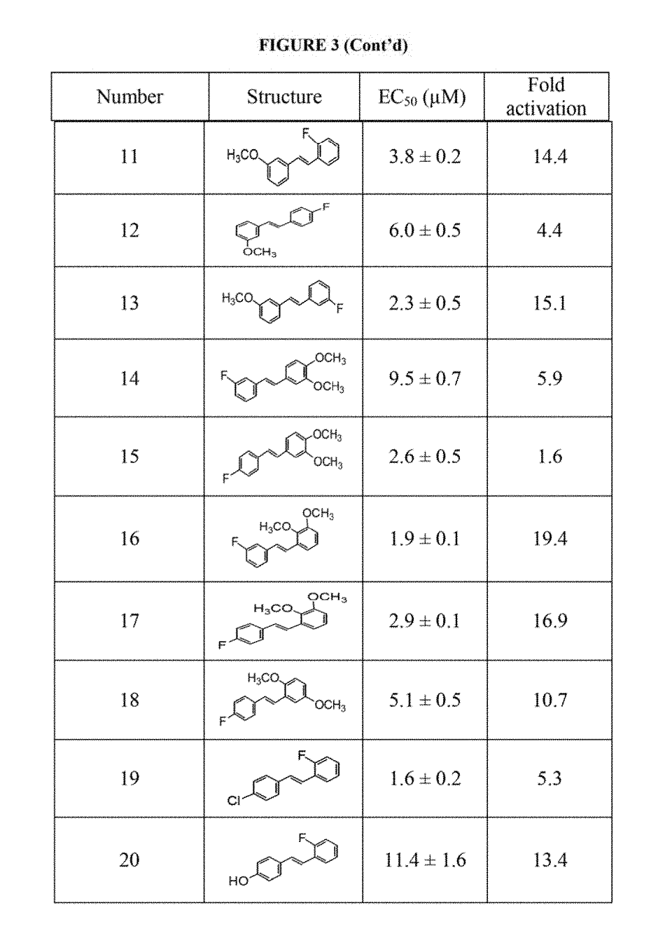

Resveratrol, LD55 and sulforaphane were compared in an Nrf2-ARE reporter-HepG2 stable cell line, which was designed for use as a luciferase reporter-based assay for activators of Nrf2. The ability of both resveratrol and LD55 to activate Nrf2 signaling extends the known activities of these two trans stilbenes to include both anti-inflammatory activities as inhibitors of the pro-inflammatory NF-κB signaling pathway as well as anti-oxidative stress activities as activators of Nrf2. It is noteworthy that the extent of activation of Nrf2 differs for these three activators (FIG. 3, Table 2). LD55 was 5-fold better than resveratrol in the extent of Nrf2 activation. Concentrations that produced 50% activation (EC₅₀) were obtained from dose-response curves. Resveratrol and LD55 show low EC₅₀ values (5.4 μM), comparable to sulforaphane (1.2 μM). The fact that these activators of Nrf2 can vary both in their EC₅₀ values and in the extent of activation requires that both of these factors be considered in evaluation of the activities of Nrf2 activators.

### 3.2. Activation of Nrf2 by Monofluoro Trans Stilbenes

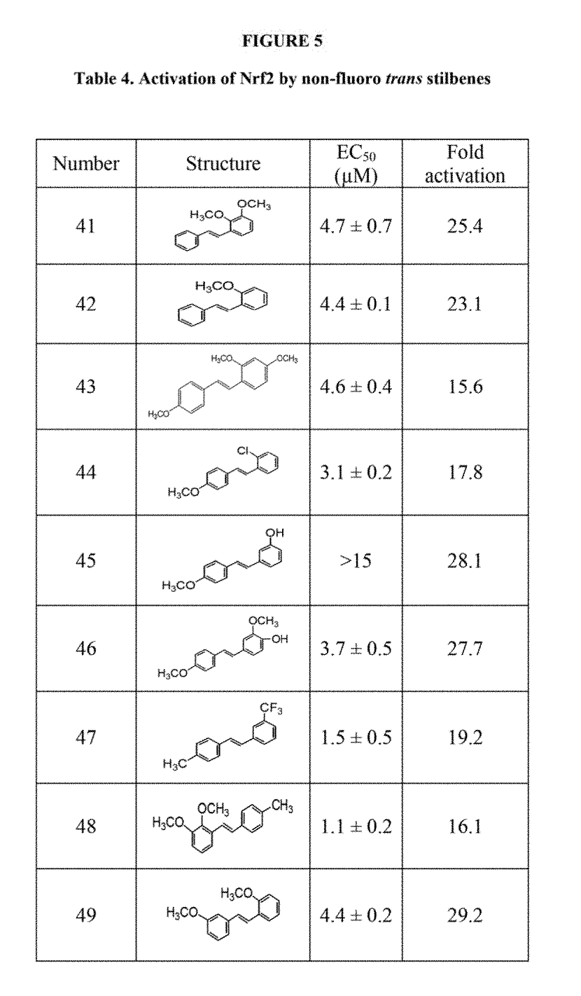

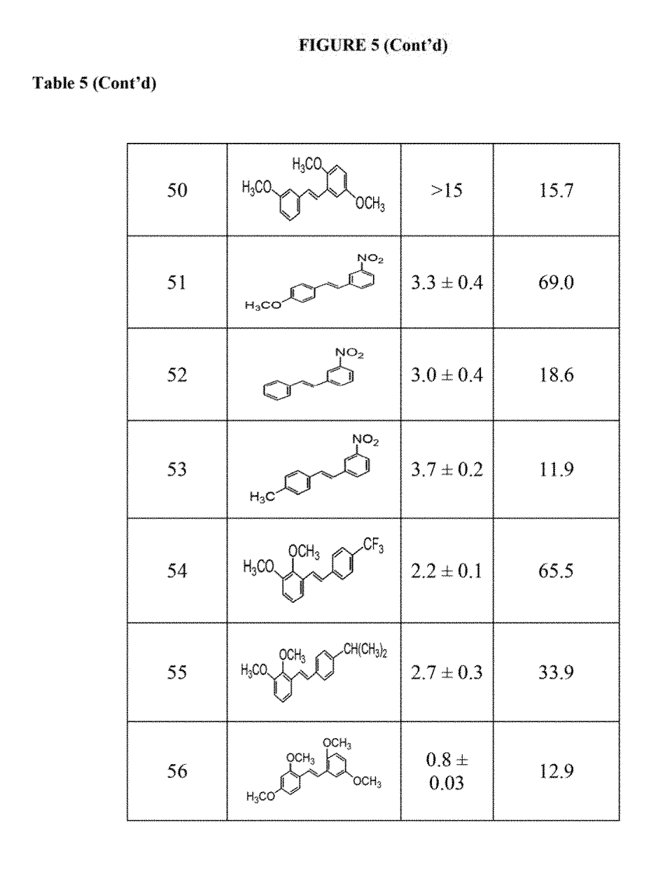

A library of substituted trans stilbenes was screened to identify activators of Nrf2. With few exceptions, such as resveratrol, only compounds with fluorine or methoxy substituents showed significant activity. From this screen, a group of 56 substituted trans stilbenes was identified as activators of Nrf2 (FIGS. 3-5, Tables 2-4). A series of monofluoro trans stilbenes was compared with resveratrol and LD55. The series was screened at 15 μM concentrations of the compounds, a concentration that did not produce any solubility problems and was sufficiently higher than the EC₅₀ values to allow a comparison of the differing extents of activation (FIGS. 3-5, Tables 2-4). All of these monofluoro trans stilbenes were activators of Nrf2, although the extent of activation varied almost 20-fold. Many of the compounds were better Nrf2 activators than sulforaphane and were much better than resveratrol. The EC₅₀ values for these monofluoro trans stilbenes ranged from 0.7 μM (trans stilbene 1) to 12.4 μM (trans stilbene 4). Comparison of trans stilbenes 3, and 4, all of which contain a single fluorine substituent on one of the aromatic rings and no substituent on the other ring, suggests that fluorine substitution in the ortho position, as in LD55, is preferred. However, this is not consistently observed. Comparison of LD55 with 5 and 6, all of which contain a para methoxy substituent, shows little effect of altering the position of the fluorine on EC₅₀ values which ranged from 4.2 to 8.9 μM (FIG. 3, Table 2). Likewise, altering the positions of both the fluorine and methoxy substituents as in LD55, 5, 6, 10-13, all of which are isomers, has a modest effect with EC₅₀ values ranging from 2.3 to 8.9 μM. The extent of activation, however, differs markedly. For example, isomer 10 is activated almost 10-fold more than isomer 6 (FIG. 3, Table 2). Replacing the methoxy substituent with a methyl substituent (7, 8 and 9) lowers EC₅₀ to low or even sub-micromolar values.

### Activation of Nrf2 by Polyfluoro Trans Stilbenes

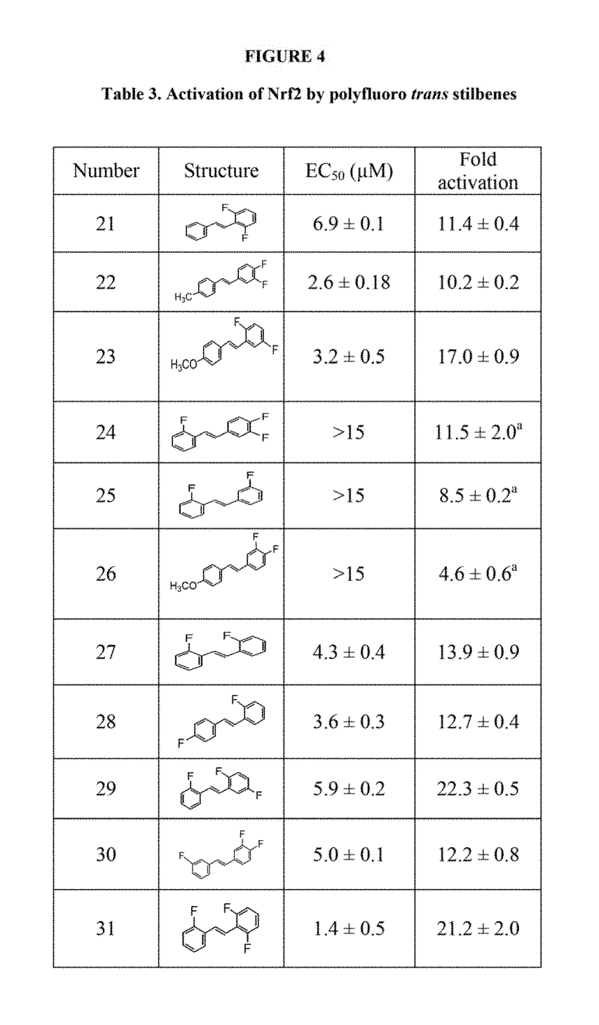

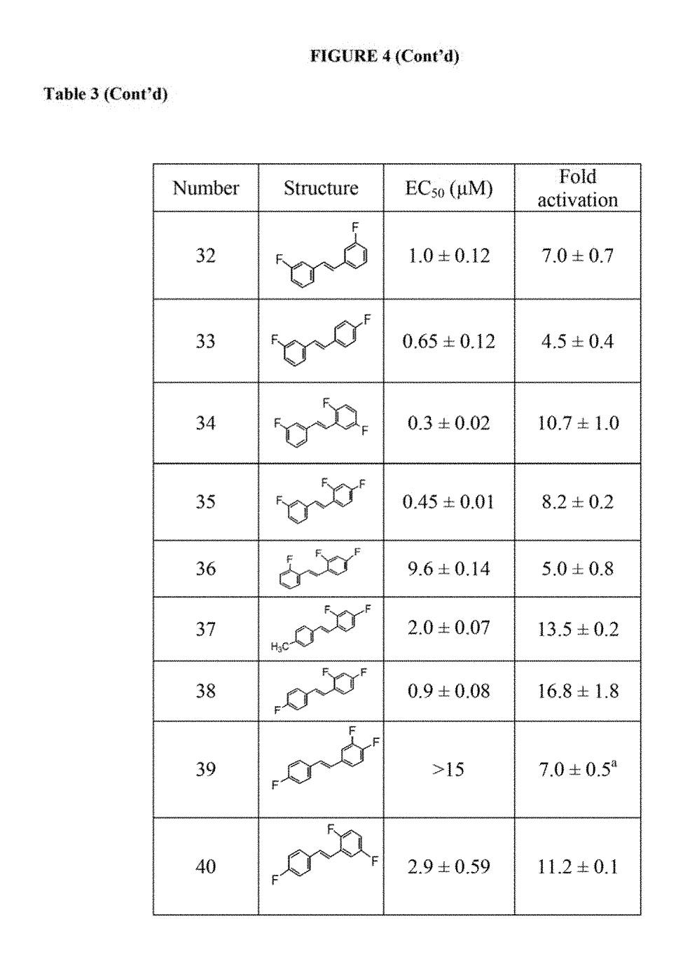

A series of polyfluoro trans stilbenes was compared with resveratrol and LD55. EC₅₀ values ranged from 0.3 to >15 μM (Table 3) and extent of activation varied about 5-fold, with all of these stilbenes better than resveratrol with respect to fold activation. As with the monofluoro trans stilbenes, the location of the fluorine substituents markedly affected EC₅₀ values and fold activations, but not in readily predictable patterns. For example, EC₅₀ for the difluoro trans stilbene 33 is sub-micromolar (0.65 μM) while EC₅₀ for its isomer 25 is >15 μM. Likewise, the EC₅₀ for the trifluoro trans stilbene 34 is sub-micromolar (0.3 μM) while EC₅₀ for its isomer 24 is >15 μM. Multiple trifluoro isomers (34, 35, 38) exhibited sub-micromolar EC₅₀ values. Isomer 34 was the most effective activator when expressed as fold activation divided by EC₅₀. The conclusion from the data in Tables 2 (FIG. 3) and 3 (FIG. 4) is that all of these fluorine substituted trans stilbenes demonstrate measurable activity as activators of Nrf2.

### Activation of Nrf2 by Nonfluoro Trans Stilbenes

A series of substituted trans stilbenes, most of which contained methoxy groups at one or more positions, demonstrated activity as activators of Nrf2, as shown in Table 4. EC₅₀ values ranged from 0.8 μM (trans stilbene 56) to >15 μM. All of these trans stilbenes were as good as or better than sulforaphane with respect to fold activation and were much better than resveratrol. Compounds 51 and 54 demonstrated the highest fold activations of all of the trans stilbenes, 69 and 65.5 fold, respectively. The results from this study support the conclusion that the stilbene scaffold is a useful structure upon which to explore chemical space to develop activators of Nrf2, especially those that include fluorine and methoxy functional groups as in LD55.

The importance of inflammation and oxidative stress in many chronic diseases supports the concept that activation of anti-oxidant Nrf2 signaling may have therapeutic potential. Cells have developed complex adaptive responses to oxidative stress to maintain redox homeostasis and to reduce oxidative stress.⁴⁵ Stress activation of Nrf2 may be the result of exposure to xenobiotics, including many natural products.⁴⁶ Many of these Nrf2 activators are electrophiles. One of the major mechanisms of Nrf2 activation is through covalent modification of select cysteine sulfhydryl residues in Keap1. Numerous Nrf2-activating chemicals, including natural products such as sulforaphane, are electrophiles that modify Keap1, often by Michael addition.⁴⁷⁻⁴⁹ Cysteine residues 273, 288 and 151 appear to be especially important targets, as determined by site-directed mutagenesis. Resveratrol and other natural product phenols activate Nrf2 after oxidation to electrophilic quinones that contain Michael acceptor functionalities.⁴⁹ Moreover, some natural product phenols including resveratrol can activate Nrf2 by additional mechanism, such as increasing the level of Nrf2 mRNA.⁴⁹

A number of Nrf2 activators have entered into clinical trials. Bardoxolone methyl, an oleanolic acid-derived synthetic triterpene, is a potent Nrf2 activator that was evaluated for its ability to slow progression to end-stage renal disease in patients with type 2 diabetes and stage 4 chronic kidney disease. Bardoxolone methyl is able to modify Keap1 as a Michael acceptor. The clinical trial was terminated in phase III owing to an increase in heart failure.⁵⁰ Dimethyl fumarate, a simple derivative of the metabolic intermediate fumaric acid, has recently been FDA-approved for the treatment of relapsing-remitting multiple sclerosis.⁵¹ Dimethyl fumarate is active as the monomethyl derivative formed by the action of non-specific esterases. Monomethyl fumarate modifies Keap1 through electrophilic addition. For both bardoxolone methyl and dimethyl fumarate, however, mechanisms in addition to modification of Keap1 may play a role in activation of Nrf2.

One concern with the design of Nrf2 activators that are electrophilic covalent modifiers of Keap1 is the issue of selectivity. Bardoxolone methyl, for example, has been shown to react with multiple targets.⁵² To address this concern, a number of recent studies have focused on development of non-electrophilic activators of Nrf2,⁵³ aided by the availability of the crystal structures of the BTB domain of Keap1, which contains residue cysteine151 that is the target for covalent modification by bardoxolone methyl,⁵⁴ the Kelch domain of Keap1⁵⁵ and the Keap1-Nrf2 interface.⁵⁶ Peptide inhibitors of the Keap1-Nrf2 protein-protein interaction have been described^{57,58} as well as a variety of small molecules that inhibit the Keap1-Nrf2 interaction.⁵⁹⁻⁶²

There are additional mechanisms for activation of Nrf2. Epigenetic modifications of CpG methylation status of Nrf2 by the anti-cancer drug 3,3′-diindolylmethane resulted in enhanced expression of Nrf2 and of Nrf2-target genes in cell-based studies.⁶³ This was suggested as an explanation for the chemopreventive activity of this drug. The promoter of the gene encoding Nrf2 contains XRE sequences, which can recruit the aromatic hydrocarbon receptor AhR; this allows the Nrf2 gene to be activated by polycyclic aromatic hydrocarbons.⁶⁴ The promoter of the Nrf2 gene also contains ARE-like sequences, which allows Nrf2 to regulate its own expression.⁶⁵ The Nrf2 promoter also contains an NF-κB binding site, which allows activation of Nrf2 by pro-inflammatory stimuli.⁶⁶

The autophagy cargo receptor and signaling adaptor protein p62 contains a binding site for Keap1. The attraction of Keap1 to this site is further regulated by phosphorylation by mTORC1, which leads to autophagosome-mediated destruction of Keap1 and activation of Nrf2.^{67,68} This is one example of proteins other than Nrf2 that interact specifically with Keap1, which indicates a broad role for Keap1 and a complex set of mechanisms that impact Nrf2 levels.⁶⁹ There are, therefore, numerous targets for potential activators of Nrf2.

In the present study, the inventors identified numerous substituted trans stilbenes as activators of Nrf2, especially trans stilbenes with fluorine and/or methoxy ring substituents. The double bond in the trans stilbene scaffold is generally a low-reactivity center which would be unlikely to modify Keap1 by electrophilic addition. It remains to be determined which site(s) is the target for activation of Nrf2 by substituted trans stilbenes. Likewise, it remains to be determined how different trans stilbenes produce markedly different fold activations, which are believed to involve e mechanisms.

### Activation of Nrf2 by Dienone Analogues of the Natural Product Curcumin

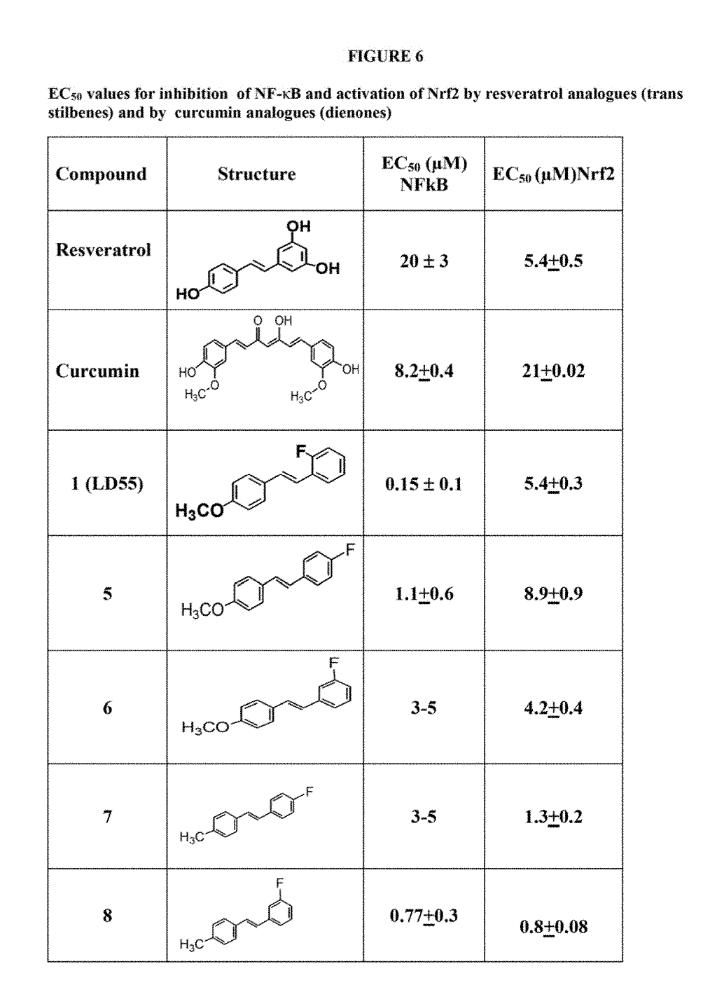

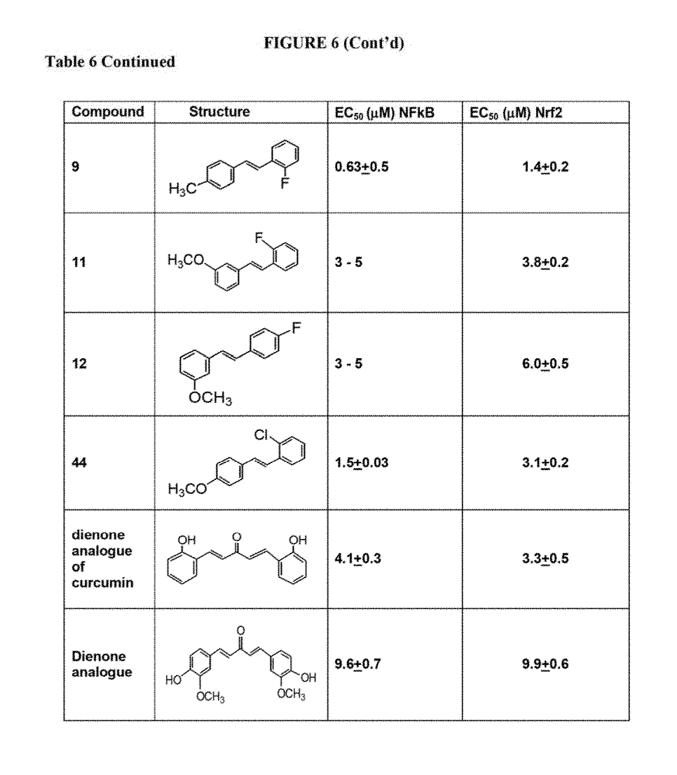

Numerous analogues of curcumin activate Nrf2. Many of these analogues also inhibit NF-κB. See, Deck, et al., European J. Med. Chem. 2018 Jan. 1; 143:854-865. However, the number of analogues of resveratrol (i.e., the trans stilbenes) and analogues of curcumin (i.e., dienones) that are both potent inhibitors of NF-κB and potent activators of Nrf2 is limited. FIG. 6 shows the structures of the trans stilbenes and dienones that are most potent as dual target analogues. All are much more potent than resveratrol or curcumin.

### CONCLUSIONS

Substituted trans stilbenes represent promising compounds for the development of drugs that target oxidative stress in chronic diseases, through activation of the anti-oxidant Nrf2 signaling pathway.

### REFERENCES

1. Guerreiro, R. J.; Hardy, J. Biochem. Soc. Trans. 2011, 39, 910.
2. Bales, K. R. Expert Opin. Drug Discov. 2012, 7, 281.
3. Fischer, O. Z. Gesamte. Psychiatr. 1910, 3, 371.
4. McGeer, E. G.; McGeer, P. L. J. Alzheimers Dis. 2010, 19, 355.
5. Alexander, J. J.; Anderson, A. J.; Barnum, S. R.; Stevens, B.; Tenner. A. J. J. Neurochem. 2008, 107, 1169.
6. Joshi, G.; Johnson, J. A. Recent Pat. CNS Drug Discov. 2012, 7, 218.
7. Sandberg, M.; Patil, J.; D'Angelo, B.; Weber, S. G.; Mallard, C. Neuropharmacology 2014, 79, 298.
8. Cardozo L. F.; Pedruzzi, L. M.; Stenvinkel, P.; Stockler-Pinto, M. B.; Daleprane, J. B.; Leite Jr, M.; Mafra, D. Biochemie 2013, 95, 1525.
9. Ruiz, S.; Pergola, P. E.; Zager, R. A.; Vaziri, N. D. Kidney Int. 2013, 83, 1029.
10. Wakabayashi, N.; Slocum, S. L.; Skoho, J. J.; Shin, S.; Kensler, T. W. Antioxid. Redox Signal. 2010, 13, 1649.
11. Ganesh Yerra, V.; Negi, G.; Sharma, S. S.; Kumar, A. Redox Biology 2013, 1, 394.
12. Hybertson, B. M.; Gao, B.; Bose, S. K.; McCord, J. M. Mol. Aspects Med. 2011, 32, 234.
13. Zhang, J.; Wang, X.; Vikash, V.; Ye, Q.; Wu, D.; Liu, Y.; Dong, W. Oxid. Med. Cell. Longev. 2016, Epub ahead of print.
14. Kumar, H.; Kim, I-S.; More, S. V.; Kim, B-W.; Choi, D-K. Nat. Prod. Rep. 2014, 31, 109.
15. Rojo, A. I.; Innamorato, N. G.; Martin-Moreno, A. M.; De Ceballos, M. L.; Yamamoto, M.; Cuadrado, A. Glia 2010.58, 588.
16. Shih, A. Y.; Li, P.; Murphy, T. H. J. Neurosci. 2005, 25, 10321.
17. Jin, W.; Wang, H.; Yan, W.; Zhu, L.; Hu, Z.; Ding, Y.; Tang, K. J. Neurotrauma 2009, 26, 131.
18. Kraft, A. D.; Johnson, D. A.; Johnson, J. A. J. Neurosci. 2004, 24, 1101.
19. Ramsey, C. P.; Glass, C. A.; Montgomery, M. B.; Lindi, K. A.; Ritson, G. P.; Chia, L. A.; Hamilton, R. L.; Chu, C. T; Jordan-Sciutto, K. L. J. Neuropathol. Exp. Neurol. 2007, 66, 75.
20. Solberg, N. O.; Chamberlin, R.; Vigil, J. R.; Deck, L. M.; Heidrich, J. E.; Brown, D. C.; Brady, C. I.; Vander Jagt, T. A.; Garwood, M.; Bisoffi, M.; Severns, V.; Vander Jagt, D. L.; Sillerud, L. O. J. Alzheimers Dis. 2014, 40, 191.
21. Heynekamp, J. J.; Weber, W. W.; Hunsaker, L. A.; Gonzales, A. M.; Orlando, R. A.; Deck, L. M.; Vander Jagt, D. L. J. Med. Chem. 2006, 49, 7182.
22. Diaz-Gerevini, G. T.; Repossi, G.; Dain, A.; Tarres, M. C.; Das, U. N.; Eynard, A. R. Nutrition 2016, 32, 174.
23. Houghton, C. A.; Fassett, R. G.; Coombes, J. S. Nutr. Rev. 2013, 71,709.
24. (a) Homer, L.; Hoffman, H.; Wippel, H. G.; Klahre, G. Chem. Ber. 1959, 92, 2499 (b) Wadsworth, W. S. Jr.; Emmons, W. D. J. Am. Chem. Soc. 1961, 83, 1733.
25. Das, J.; Pany, S.; Majhi, A. Bioorg. Med. Chem. 2011, 19, 5321
26. Arbuzov, B. A. Pure Appl. Chem. 1964, 9, 307.
27. Rivera, H.; Morales-Rios, M. S.; Bautista, W.; Shibayama, M.; Tsutsumi, V.; Muriel, P.; Perez-Alvarez, V. Can. J. Physiol. Pharmacol. 2011, 89, 759.
28. Yunes, S.; Tchani, G.; Baziard-Mouysset, G.; Stigliani, J. L.; Payard, M.; Bonnafous, R.; Tisne-Versailles, J. Eur. J. Med. Chem. 1994, 29, 87.
29. Zhang, X.; Zeng, W.; Yang, Y.; Huang, H.; Liang, Y. Synlett 2013, 24, 1687
30. Gust, R.; Schoenenberger, H. Arch. Pharmazie, 1995, 328, 595.
31. Cao, C.; Sheng, B.; Chen, G. J. Phys. Org. Chem Gust, R.; Schoenenberger, H. Arch. Pharmazie. 2012, 25, 1315.
32. Kang, S.; Cuendet, M.; Endringer, D. C.; Croy, V. L.; Pezzieto, J. M.; Lipton, M. A. Bioorg. Med. Chem. 2009, 17, 1044.
33. Sun, B.; Hoshino, J.; Jermihov, K.; Marlero, L.; Pezzuto, J. M.; Mesecar, A. D.; Cushman, M. Bioorg. Med. Chem. 2010, 18, 5352.
34. Lion, C.; Matthews, C. S.; Stevens, M. F. G.; Westwell, A. D. J. Med. Chem. 2005, 48, 1292.
35. Dunne, E. C.; Coyne, E. J.; Crowley, P. B.; Gilheany, D. G. Tetrahedron Lett. 2002, 43, 2449.
36. Buu-Hoi, N. P.; Xuong, N. D.; Diep, B. K.; Quang, N. N. J. Org. Chem. 1962, 27, 2669.
37. Zhang, M.; Jia, T.; Sagamanova, I. K.; Pericas, M. A.; Walsh, P. J. Org. Lett. 2015, 17, 1164.
38. Karki, S. S.; Bhutle, S. R.; Sahoo, S.; Reddy, R.; Balzarini, J.; De Clercq, E.; Darji, S. Y. Med. Chem. Res. 2011, 20, 1349.
39. Karke, S. S.; Bhutle, S. R.; Pedgaonkar, G. S.; Zubaidha, P. K.; Shaikh, R. M.; Rajput, C. G.; Shendarkar, G. S. Med. Chem. Res. 2011, 20, 1158.
40. Chalal, M.; Vervandier-Fasseur, D.; Meunier, P.; Cattey, H.; Hierso, J-C. Tetrahedron 2012, 68, 3899.
41. Ao, J.; Chen, Y.; Xu, X.; Zhang, X.; Yu, Y.; Yu, P.; Hua, E. Asian J. Chem. 2014, 26, 2092.
42. Saiyed, A. S.; Patel, K. N.; Kamath, B. V.; Bedekar, A. V. Tetrahedron Lett. 2012, 53, 4692.
43. Motoshima, K.; Sugita, K.; Hashimoto, Y.; Ishikawa, M. Bioorg. Med. Chem. Lett. 2011, 21, 3041.
44. Khandelwal, M.; Hwang, I.; Nair, P. C.; Lee, J-W. Bull. Korean Chem. Soc. 2012, 33, 1190.
45. Hayes, J. D.; Dinkova-Kostova, A. T. Trends Biochem. Sci. 2014, 39, 199.
46. Kuma, H.; Kim, I-S.; More, S. V.; Kim, B-W.; Choi, D-K. Nat. Prod. Rep. 2014, 31, 109.
47. Zhang, D. D.; Hannink, M. Mol. Cell. Biol. 2003, 23, 8137.
48. Keum, Y-S.; Choi, B. Y. Molecules 2014, 19, 10074.
49. Magesh, S.; Chen, Y.; Hu, L. Med. Res. Rev. 2012, 32, 687.
50. Chin, M. P.; Wrolstad, D.; Bakris, G. L.; Chertpw, G. M.; De Zeeuw, D.; Goldberry, A.; Linde, P. G.; McCullough, P. A.; McMurry, J. J.; Wittes, J.; Meyer, C. J. J. Cardiac Fail. 2014, 20, 953.
51. Bomprezri, R. Ther. Adv. Neurol. Disord. 2015, 8, 20.
52. Yore, M. M.; Kettenbach, A. N.; Sporn, M. B.; Gerber, S. A.; Liby, K. T. PLoS One, 2011, 6, e22862.
53. Richardson, B. G.; Jain, A. D.; Speltz, T. E.; Moore, T. W. Bioorg. Med. Chem. Lett. 2015, 25, 2261.
54. Cleasby, A.; Yon, J.; Day, P. J.; Richardson, C.; Tickle, I. J.; Williams, P. A.; Callahan, J. F.; Carr, R.; Concha, N.; Kerns, J. K.; Qi, H.; Sweitzer, T.; Ward, P. Davies, T. G. PLoS One 2014, 9, e98896.
55. Li, X.; Zhang, D.; Hannink, M.; Beamer, L. J. J. Biol. Chem. 2004, 24, 279.
56. Lo, S. C; Li, X.; Henzl. M. T.; Beamer, L. J.; Hannink. M. EMBO J. 2006, 25, 3605.
57. Hancock, R.; Bertrand, H. C.; Tsujita, T.; Naz, S.; EI-Bakry, A.; Laoruchupong, J.; Hayes, J. D.; Wells, G. Free Radic. Biol. Med. 2012, 52, 444.
58. Steel, R.; Cowan, J.; Payerne, E.; O'Connell, M. A.; Searcey, M. ACS Med. Chem. Lett. 2012, 3, 407.
59. Hu, L.; Magesh, S.; Chen, L.; Lewis, T. A.; Chen, Y.; Khodier, C.; Inoyama, D.; Beamer, L. J.; Emge. T. J.; Shen, J.; Kerrigan, J. E.; Kong, A. N.; Dandapani, S.; Palmer, M.; Schreiber, S. L.; Munoz, B. Bioorg. Med. Chem. Lett. 2013, 23, 3039.
60. Jnoff, E.; Albrecht, C.; Barker, J. J.; Barker, O. Beaumont, E.; Bromidge, S.; Brookfield, F.; Brooks, M.; Bubert, C.; Ceska, T.; Corden, V.; Dawson, G.; Duclos, S.; Fryatt, T.; Genicot, C. Jigorel, E.; Kwong, J.; Maghames, R.; Mushi, I.; Pike, R.; Sands, Z. A.; Smith, M. A.; Stimson, C. C.; Courade, J. P. ChemMedChem. 2014, 9, 699.
61. Marcotte, D.; Zeng, W.; Hus, J-C.; McKenzie, A.; Hession, C.; Jin, P.; Bergeron, C.; Lugovskoy, A.; Enyedy, I.; Cuervo, H.; Wang, D. Atmanene, C.; Roecklin, D.; Vecchi, M.; Vivat, V.; Kraemer, J.; Winkler, D.; Hong, V.; Chao, J.; Lukashev, M.; Silvian, L. Biorg. Med. Chem. 2013, 21, 4011.
62. Sun, H-P.; Jiang, Z-Y.; Zhang, M-Y.; Lu, M-C.; Yang, T-T.; Pan, Y.; Huang, H-Z.; Zhang, X-J.; You, Q-D. MedChemComm. 2014, 5, 93.
63. Wu, T. Y.; Khor, T. O.; Su, Z. Y.; Saw, C. L.; Shu, L.; Cheung, K. L.; Huang, Y.; Yu, S.; Kong, A. N. AAPS J. 2013, 15, 864.
64. Ma, Q.; Kinneer, K.; Bi, Y.; Chan, J. Y.; Kan, Y. W. Biochem. J. 2004, 377, 205.
65. Kwak, M-K.; Itoh, K.; Yamamoto, M.; Kensler, T. W. Mol. Cell. Biol. 2002, 22, 2883.
66. Rushworth, S. A.; Zaitseva, L.; Murray, M. Y.; Shah, N. M.; Bowles, K. M.; MacEwan, D. J. Blood 2012, 120, 5188.
67. Lau, A.; Wang, X-J.; Zhao, F.; Villeneuve, N. F.; Wu, T.; Jiang, T.; Sun, Z.; White. E.; Zhang, D. D. Mol. Cell. Biol. 2010, 30, 3275.
68. Ichimura, Y.; Waguri, S.; Sou, Y.; Kageyama, S.; Hasegawa, J.; Ishimura, R.; Saito, T.; Yang, Y.; Kouno, T.; Fukutomo, T.; Hoshii, T.; Hirao, A.; Takagi, K.; Mizushima, T.; Motobashi, H.; Lee, M. S.; Yoshimori, T., Tanaka, K.; Yamamoto, M.; Komatsu, M. Mol. Cell 2013, 51, 618.
69. Tebay, L. E.; Robertson, H.; Durant, S. T.; Vitale, S. R.; Penning, T. M.; Dinkova-Kostova, A. T.; Hayes, J. D. Free Radic. Biol. Med. 2015, 88, 108.

1. A method of a treating a subject afflicted with a disease or condition which is modulated through upregulation of NF-κB signaling and inhibition of Nrf2 signalling, the method comprising administering to the subject a composition comprising a therapeutically effective amount of at least one compound which exhibits dual activity as a NF-κB signaling inhibitor and a Nrf2 signaling agonist, optionally in combination with at least one additional bioactive agent.

2. The method of claim 1, wherein the composition further comprises a pharmaceutically acceptable carrier.

3. The method of claim 1, wherein the compound is according to the chemical structure: 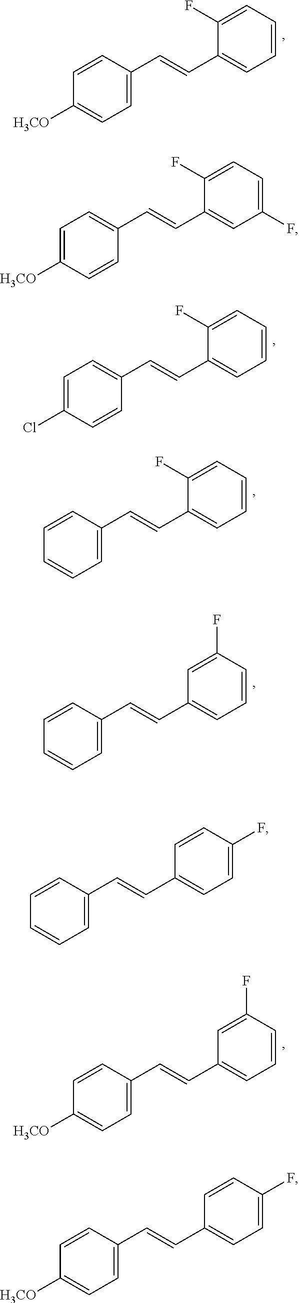 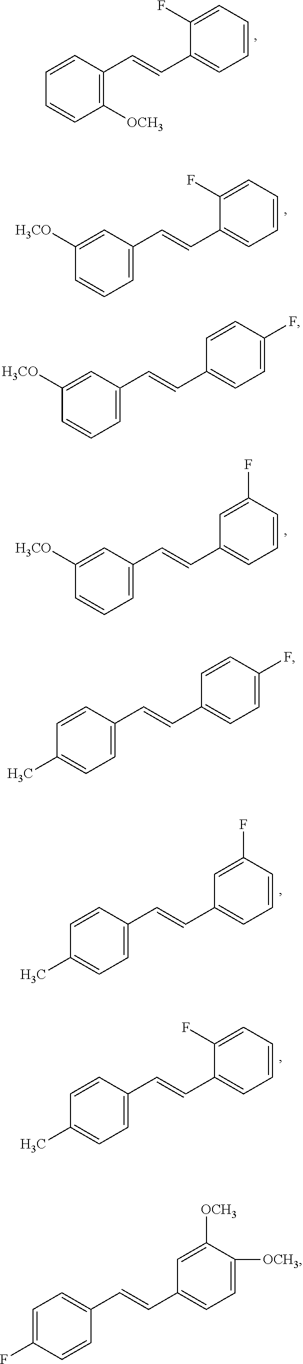 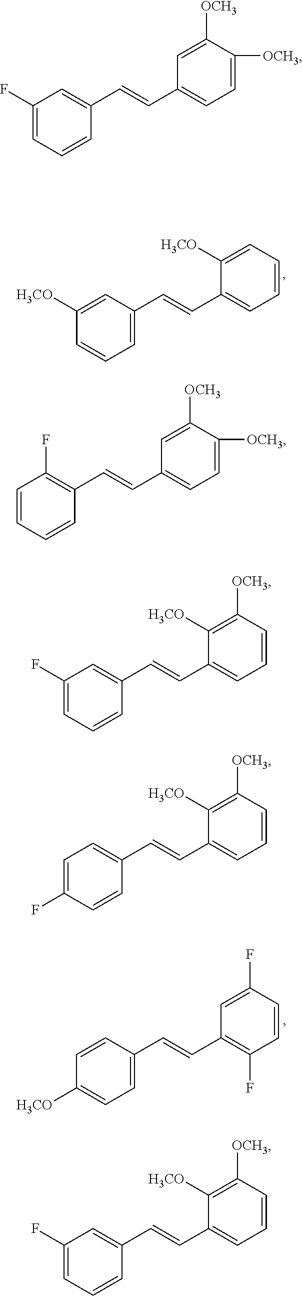 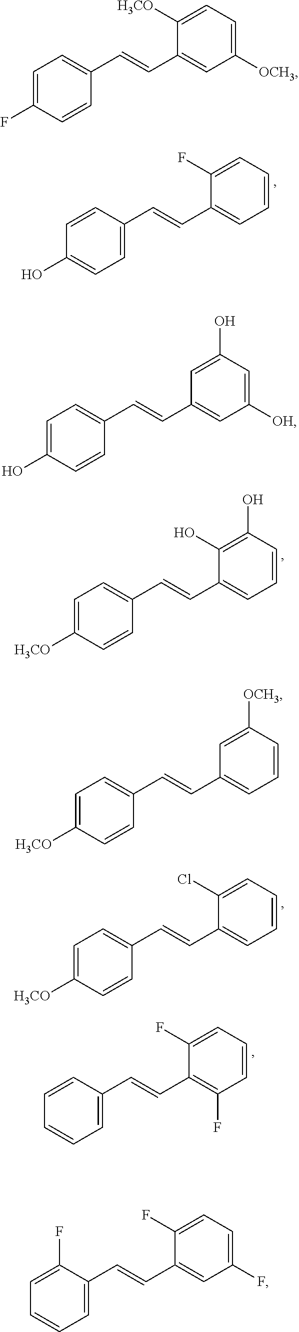 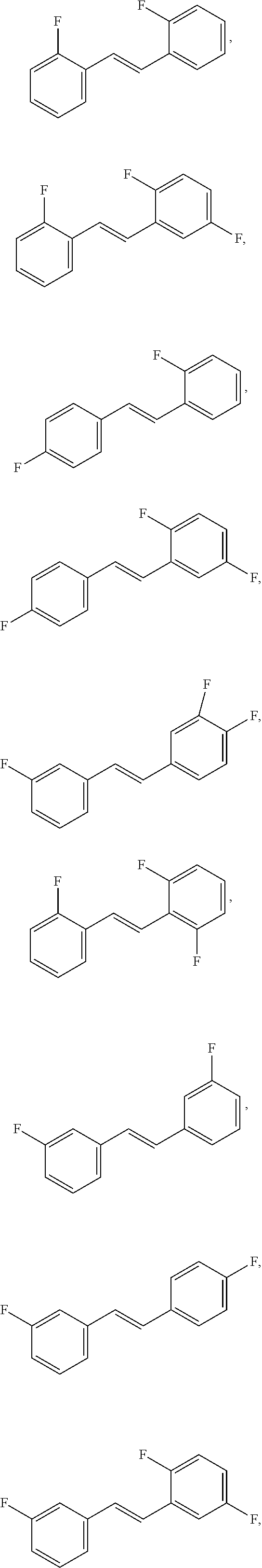 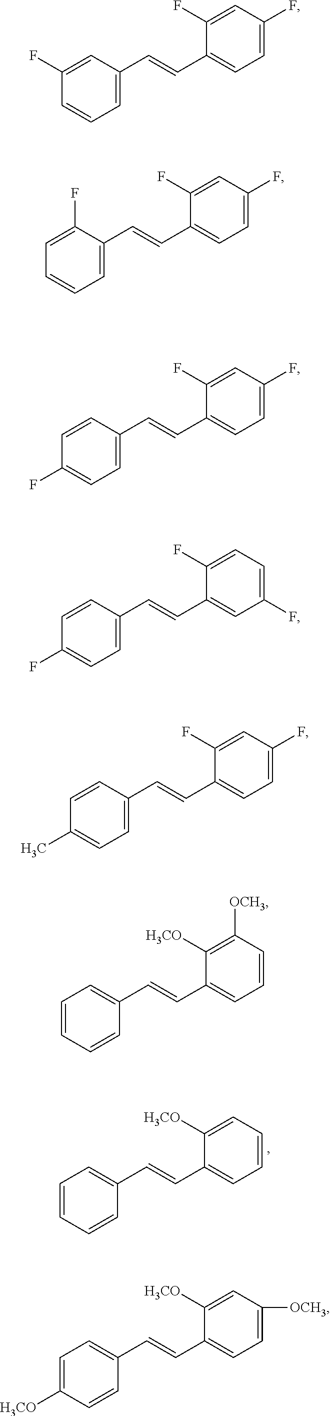 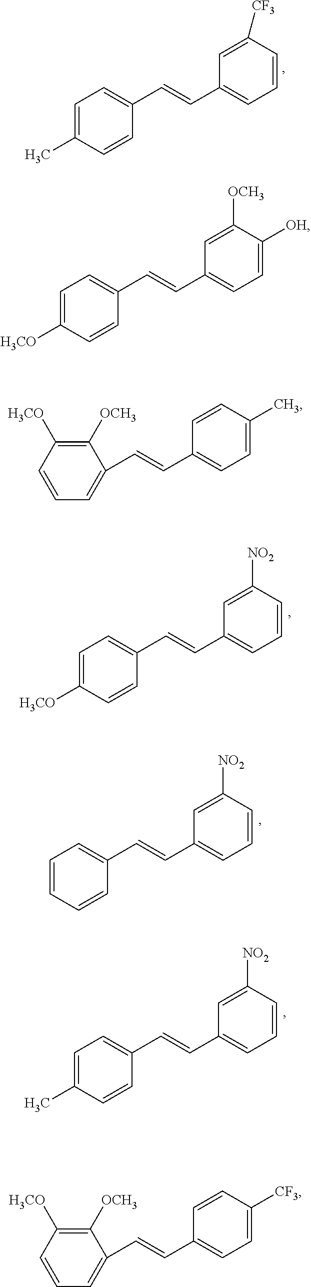 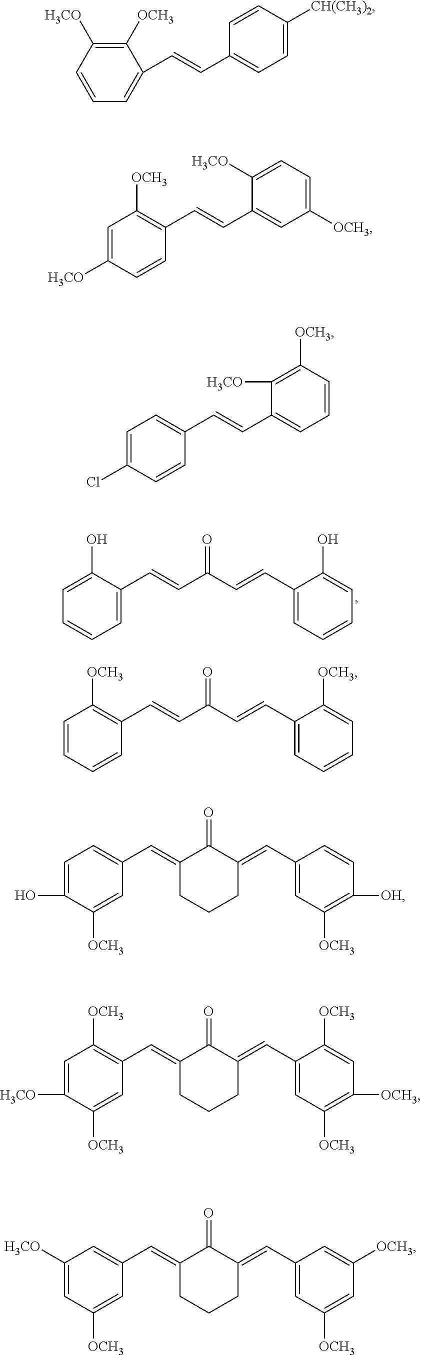 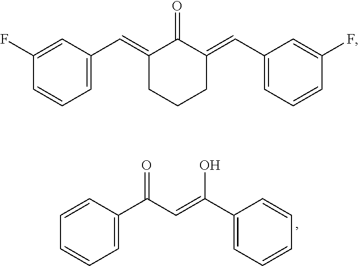 [SMILES: O=C1/C(=C/c2cccc(F)c2)CCC/C1=C\c1cccc(F)c1 ; O=C(/C=C(\O)c1ccccc1)c1ccccc1]
a pharmaceutically acceptable salt thereof, or a mixture thereof.

4. The method according to claim 3 wherein said compound(s) is 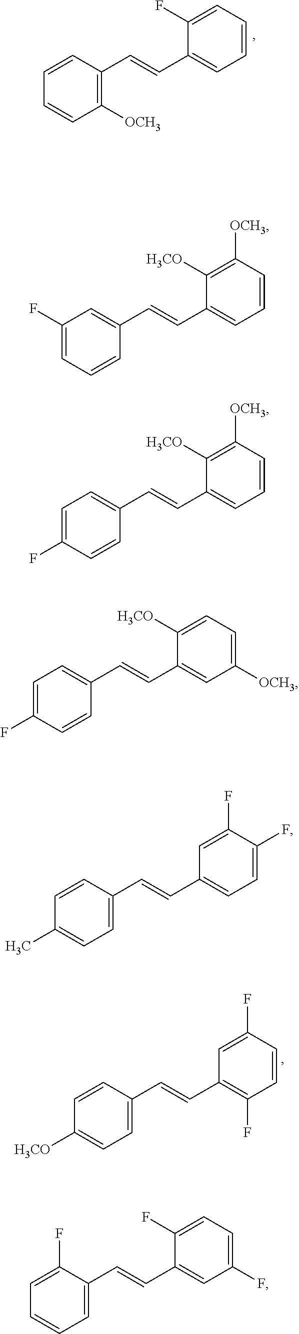 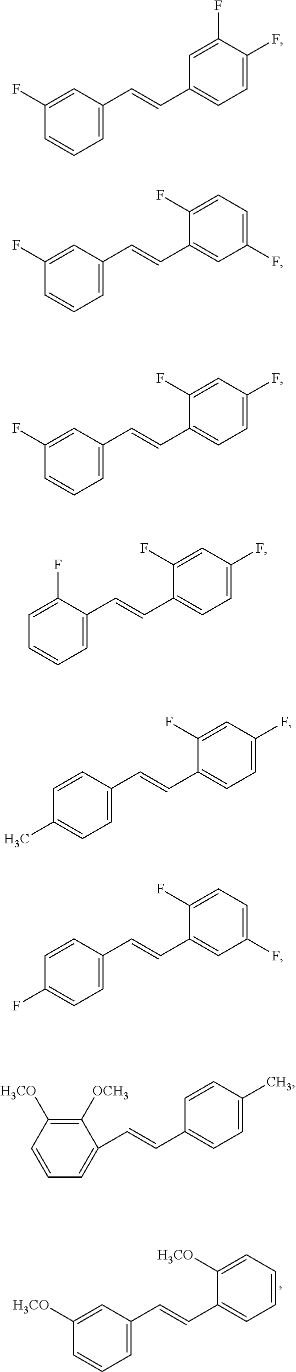 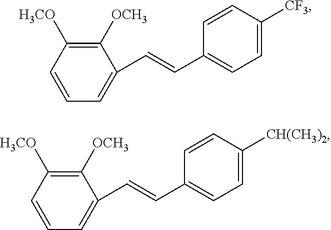
a pharmaceutically acceptable salt thereof or a mixture thereof.

5. The method according to claim 1 wherein said compound is 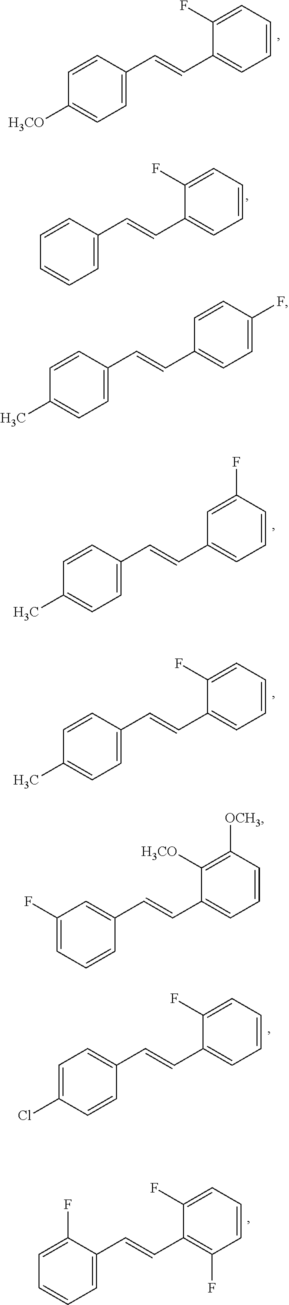 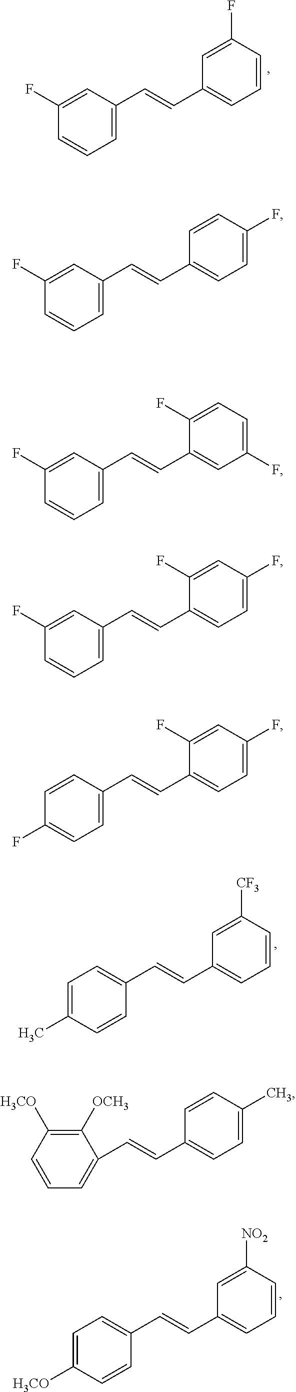 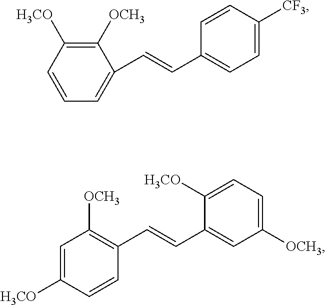 [SMILES: COc1cccc(/C=C/c2ccc(C(F)(F)F)cc2)c1OC ; COc1ccc(OC)c(/C=C/c2ccc(OC)cc2OC)c1]
or
a pharmaceutically acceptable salt thereof or mixture thereof.

6. The method according to claim 1 wherein said compound is administered in combination with at least one additional bioactive agent.

7. The method according to claim 1 wherein said disease or condition is Alzheimer's; ALS; autism; bipolar disorder; brain injury; chronic pain; chronic inflammatory demyelinating polyneuropathy (CIPD); diabetic neuropathy; epilepsy; fibromyalgia; Huntington's; inflammatory myopathy; meningitis; migraine; multiple sclerosis (MS); Parkinson's; stroke; chronic kidney disease; Crohn's; diabetes mellitus type 1 and type 2; rheumatoid arthritis, osteoarthritis, psoriatic arthritis, lupus, gout; ulcerative colitis; acne; eczema; psoriasis; tendinitis; atherosclerosis; obesity; cancer; allergies or depression.

8. The method according to claim 1 wherein said disease state or condition is a skin disease.

9. The method according to claim 8 wherein said skin disease or condition is Acrodermatitis, Cellulite, Cryotherapy, Cutaneous skin tags, Dermatitis herpetiformis, Dry skin, Ectodermal dysplasia, Epidermolysis bullosa, Erythema multiforme, Erythema nodosum, Erythema toxicum, Granuloma annulare, Henoch-Schonlein purpura, Hyperelastic skin, Ichthyosis vulgaris, Idiopathic or primary livedo reticularis, Intertrigo, Keratosis pilaris, Lamellar ichthyosis, Lichen planus, Lichen simplex chronicus, Milia, Nikolsky's sign, Perioral dermatitis, Pityriasis rosea, Pityriasis rubra pilaris, Polymorphic light eruption, Preauricular tag or pit, Purpura Pyogenic granuloma, Sebaceous cyst, Seborrheic dermatitis, Seborrheic keratosis, Skin and hair changes during pregnancy, Skin blushing/flushing, Skin discoloration—bluish, Skin graft, Skin lesion biopsy, Skin lumps, Skin turgor, Stasis dermatitis and ulcers, Striae, Subcutaneous emphysema, Vesicles, Wood's lamp examination, Xanthoma, Xeroderma pigmentosa, Xerosis, Eczema, Impetigo, Itching, Psoriasis, Rashes, Scleroderma, Skin Aging, Skin Cancer, Skin Infections or a Skin Pigmentation Disorder.

10. The method according to claim 7 wherein said disease state or condition is cancer.

11. The method according to claim 10 wherein said cancer is carcinoma (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, thyroid, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas, medulloblastoma and B-cell lymphoma.

12. The method according to claim 10 wherein said compound is co-administered with an additional anti-cancer agent.

13. The method according to claim 12 wherein said additional anti-cancer agent is everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-1-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SUI 1248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac (imitinib), hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa orarbepoetin alfa, ipilimumab, pembrolizumab, nivolumab, alemtuzumab, brentuximab vedotin, blinatumomab, cetuximab or a mixture thereof.

14. The method according to claim 1 wherein said compound is coadministered with resveratrol.

15-29. (canceled)

30. A compound according to the chemical structure: 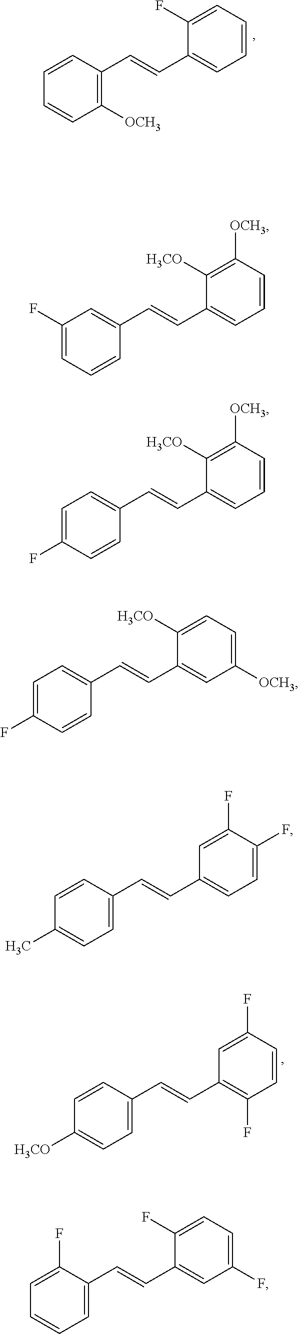 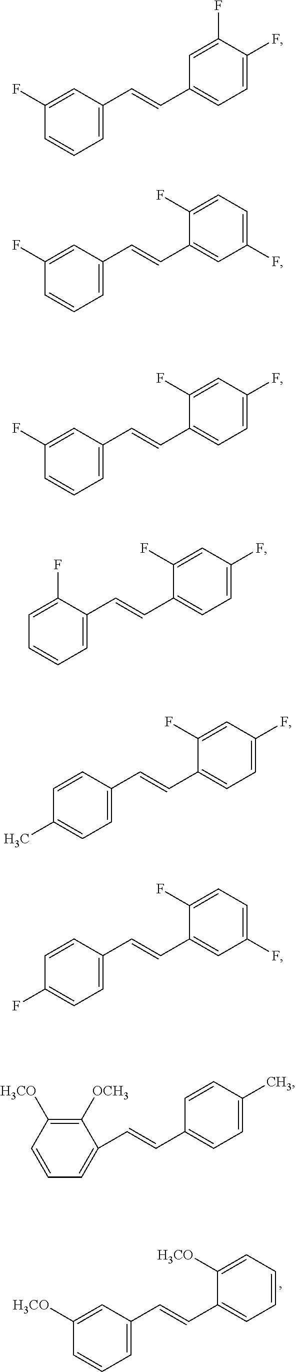 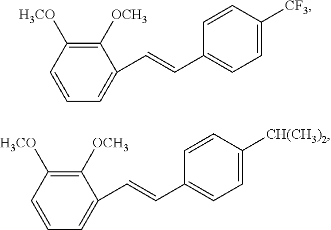
or a pharmaceutically acceptable salt thereof.

31. A pharmaceutical composition comprising an effective amount of a compound according to claim 30 in combination with a pharmaceutically acceptable carrier, additive and/or excipient.

32. A pharmaceutical composition comprising an effective amount of a compound according to the chemical structure: 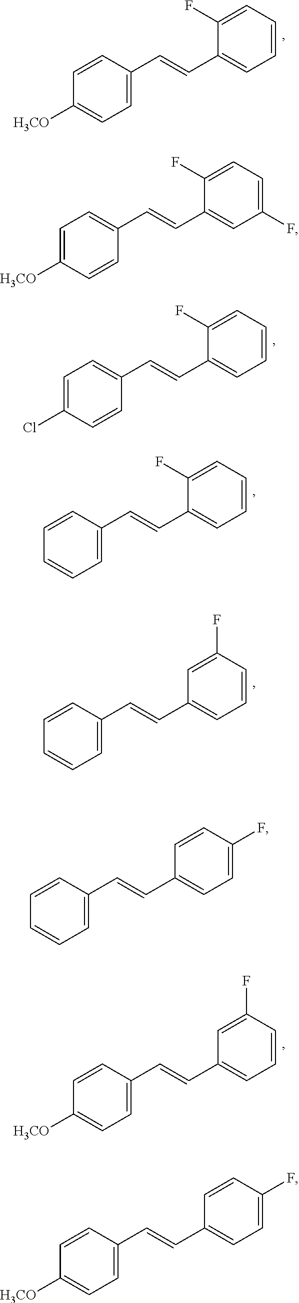 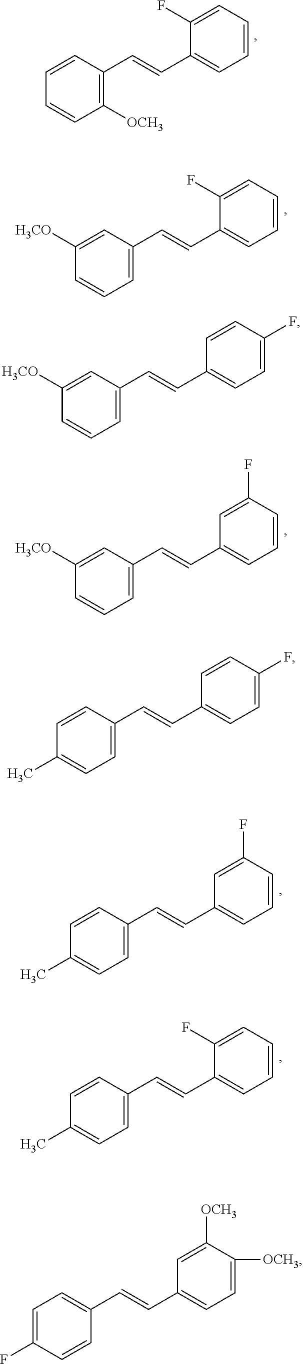 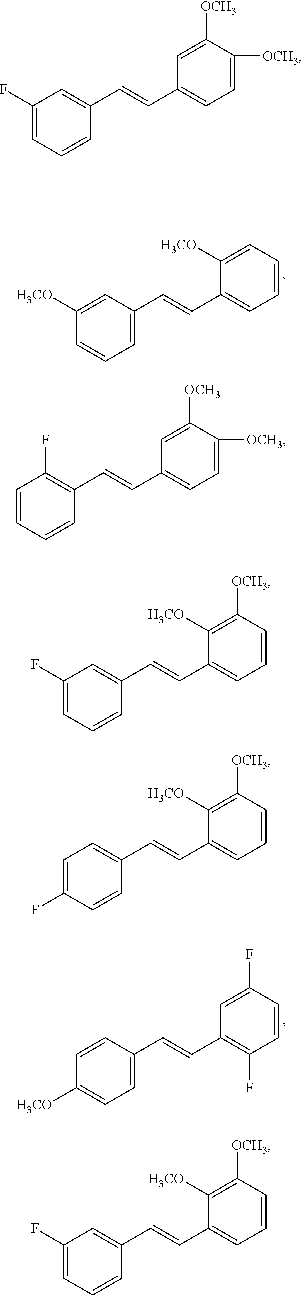 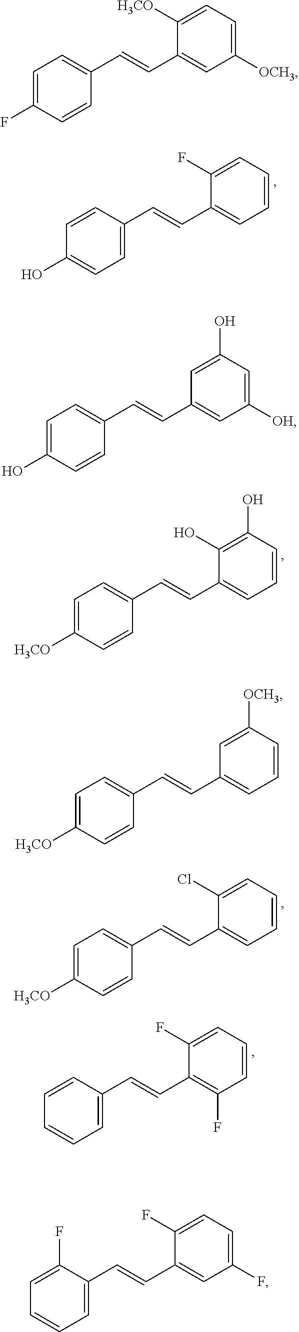 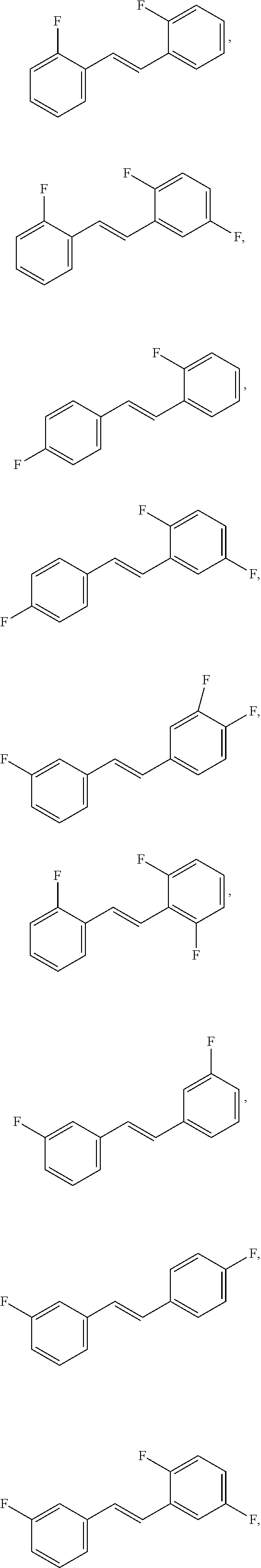 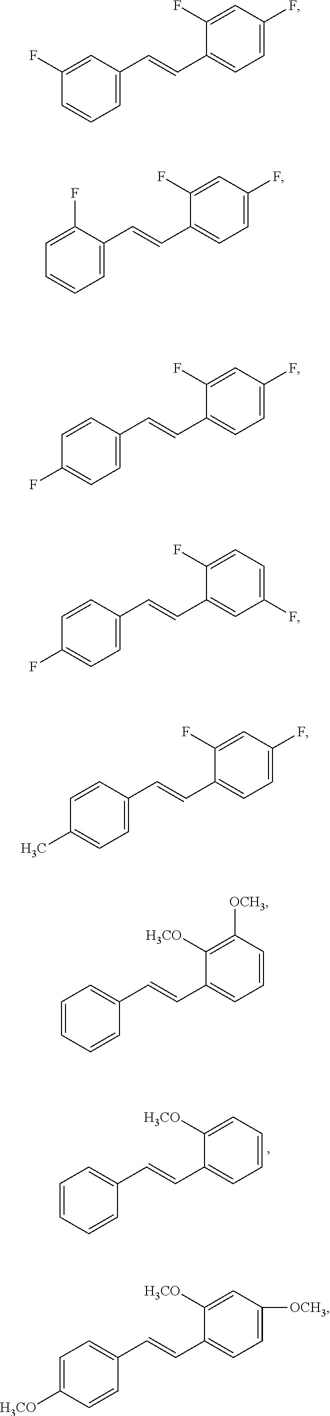 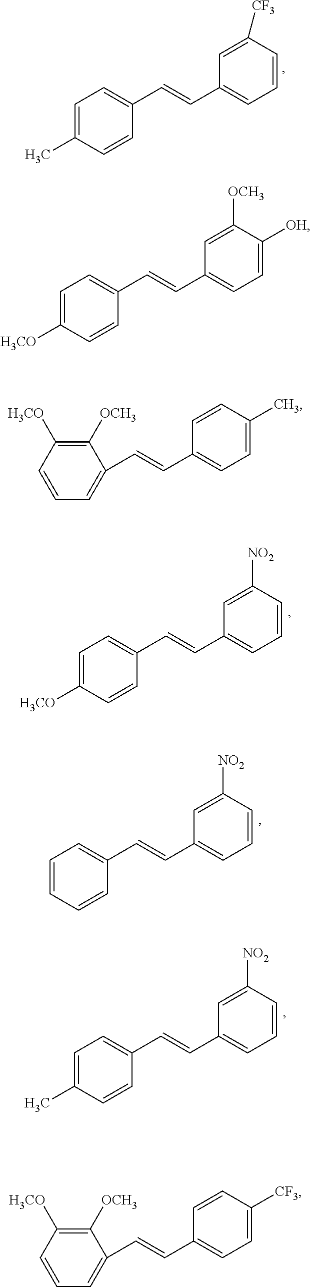 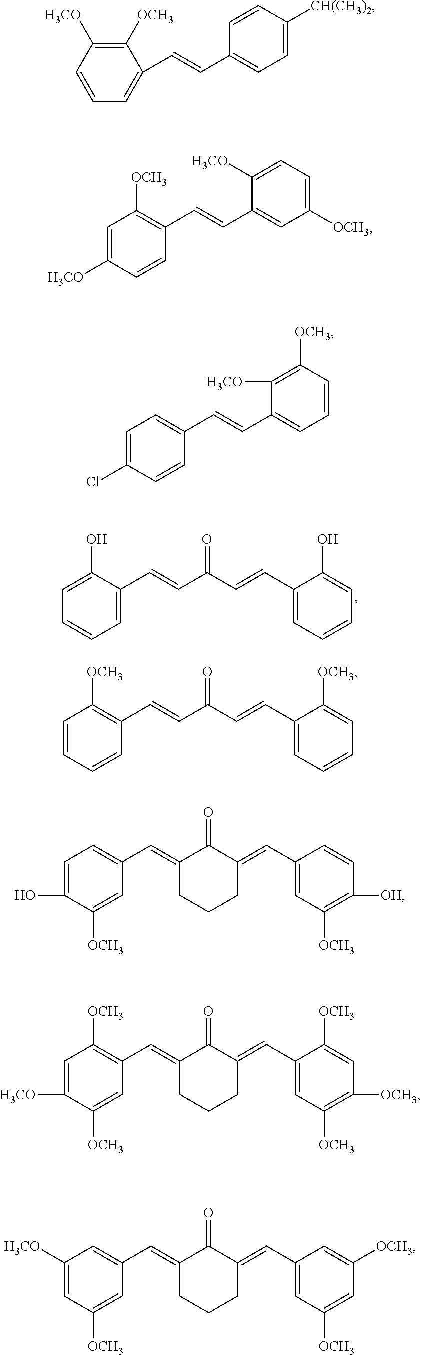 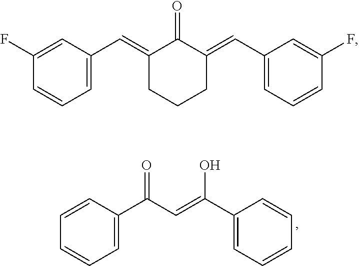 [SMILES: O=C1/C(=C/c2cccc(F)c2)CCC/C1=C\c1cccc(F)c1 ; O=C(/C=C(\O)c1ccccc1)c1ccccc1]
or
a pharmaceutically acceptable salt thereof, or a mixture thereof in combination with a pharmaceutically acceptable carrier, additive and/or excipient.

33. The composition according to claim 32 further comprising an additional bioactive agent.

34. The composition according to claim 33 wherein said bioactive agent is an additional anti-cancer agent.

35. The composition according to claim 34 wherein said additional anticancer agent is everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-1-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SUI 1248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac (imitinib), hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 3-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-1, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, arbepoetin alfa, ipilimumab, pembrolizumab, nivolumab, alemtuzumab, brentuximab vedotin, blinatumomab, cetuximab or a mixture thereof.
